(19) United States
(12) Patent Application Publication
(10) Pub. No.: US 2008/0207650 A1
(43) Pub. Date: Aug. 28, 2008

(54) Chemical Compounds 636

(76) Inventors: Roger Victor Bonnert, Loughborough (GB); Hitesh Jayantilal Sanganee, Loughborough (GB)

Correspondence Address: FISH & RICHARDSON P.C., P.O BOX 1022, MINNEAPOLIS, MN, 55440-1022, US

(21) Appl. No.: 11/972,219

(22) Filed: Jan. 10, 2008

Related U.S. Application Data
(60) Provisional application No. 60/884,455, filed on Jan. 11, 2007.
(60) Provisional application No. 60/952,049, filed on Jul. 26, 2007.

(51) Int. Cl. A61K 31/519; C07D 471/04

(52) U.S. Cl. 514/264.1; 544/279

(57) ABSTRACT

The present invention provides a compound of a formula (I):

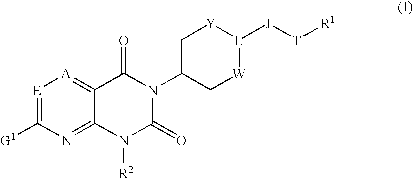

wherein the variables are defined herein; to a process for preparing such a compound; and to the use of such a compound in the treatment of a PDE 4 mediated disease state.

The present invention concerns pyridopyrimidine derivatives having pharmaceutical activity, to processes for preparing such derivatives, to pharmaceutical compositions comprising such derivatives and to the use of such derivatives as active therapeutic agents.

Pharmaceutically active pyridopyrimidine derivatives are disclosed in EP-A-0260817, WO 98/02162, WO 93/19068 and WO 0045800.

Phosphodiesterases (PDEs) work by converting cAMP or cGMP to AMP and GMP, or the inactive nucleotide forms incapable of activating downstream signalling pathways. The inhibition of PDEs leads to the accumulation of cAMP or cGMP, and subsequent activation of downstream pathways. PDEs comprise a large family of second messengers with 11 families and over 50 isoforms. In addition splice variants have been described for each isoform. The PDEs can be cAMP-specific (PDE4, 7, 8, 10), cGMP specific (PDE5, 6, 9) or have dual specificity (PDE1, 2, 3, 11).

cAMP is generated from ATP at the inner leaflet of the plasma membrane through the action of GPCR-regulated adenylate cyclase. Once cAMP is generated, the only way to terminate the signal is through phosphodiesterase action, degrading cAMP into 5′-AMP. Increased concentrations of cAMP are translated into cellular responses mainly by activation of cAMP-dependent protein kinase (PKA). The specific activity of PKA is in part regulated by the sub-cellular localization of PKA, which limits the phosphorylation of PKA to substrates in its near vicinity. The downstream events caused by activation of PKA appear poorly elucidated and involve many components in the initiation of signalling cascades. PDE4s have been shown to have abundant roles in regulating cell desensitisation, adaptation, signal cross-talk, cAMP compartmentalization and feedback loops, and are major regulators of cAMP homeostasis.

The physiological role implicated for elevated cAMP levels include: 1) broad suppression the activity of many immunocompetent cells; 2) induction of airway smooth muscle relaxation; 3) suppression of smooth muscle mitogenesis; and, 4) has beneficial modulatory effects on the activity of pulmonary nerves.

PDE4 has been found to be the predominant cAMP metabolising isozyme family in immune and inflammatory cells and, along with the PDE3 family, a major contributor to cAMP metabolism in airway smooth muscle.

Over the last two decades significant attention has been devoted into the development of PDE4 selective inhibitors for the treatment of inflammatory and immune disorders including asthma, rhinitis, bronchitis, COPD, arthritis and psoriasis. A number of compounds (for example rolipram, tibenelast and denbufylline) have been reported to have impressive effects in animal models of inflammation, especially pulmonary inflammation.

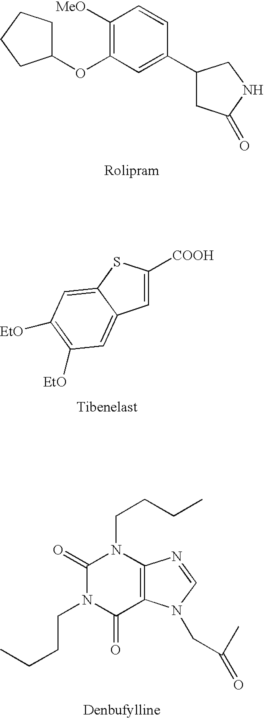

Unfortunately the clinical utility of these inhibitors has been limited by PDE4 related side-effects, including nausea, vomiting and gastric acid secretion. Recently a second generation of PDE4 inhibitors (for example cilomilast, roflumilast and AWD 12-281) has been described having significantly reduced risk of emetic side effects in animal models of emesis, thus providing the potential for an increased therapeutic ratio.

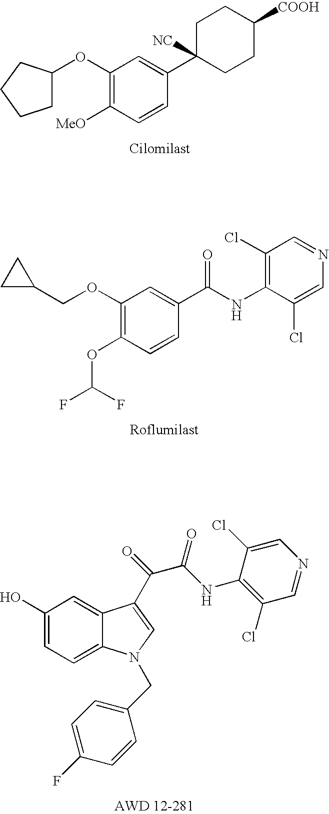

The present invention discloses novel pyridopyrimidine derivatives that are inhibitors of human PDE4 and are thereby useful in therapy.

The present invention provides a compound of formula (I):

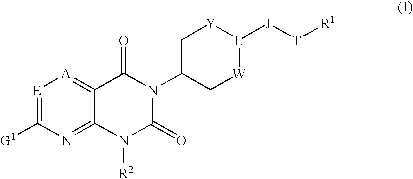

wherein:

### E is N or CE¹;

### A is N or CA¹;

### T is C(O) or S(O)₂;

W is (CH₂)ₙ;
Y is (CH₂)ₚ;
n and p are, independently 0 or 1;
R² is tetrahydrothiopyran-4-yl, tetrahydrothiopyran-4-yl S-oxide, tetrahydrothiopyran-4-yl S-dioxide, tetrahydrothiopyran-4-yl, tetrahydrothiopyran-4-yl S-oxide or tetrahydrothiopyran-4-yl S-dioxide; or aryl or heteroaryl either of which is substituted by one or more of S(O)aryl, S(O)₂aryl, NR²⁵COR²⁶, CONR²⁷R²⁸ (but not C(O)NHaryl), S(O)₂NR²⁹R³⁰ or NRS(O)₂R³¹, and either of which may be additionally optionally substituted by halogen, cyano, hydroxy, C₁₋₄ alkyl, C₁₋₄ alkoxy, CF₃, OCF₃, C₁₋₄ alkylthio, S(O)(C₁₋₄ alkyl), S(O)₂(C₁₋₄ alkyl) or CO₂(C₁₋₄ alkyl);
L is CH or N; when L is CH then J is NH; and when L is N then J is absent and T is bonded directly to L; AND
R¹ is C₁₋₆ alkyl {substituted by either NR³R⁴ or nitrogen-containing heterocyclyl; and optionally additionally substituted by aryl, heteroaryl, C₃₋₇ cycloalkyl, C₃₋₇ cycloalkyl(C₁₋₄ alkyl), aryl(C₁₋₄alkoxy), aryl(C₁₋₄ alkylthio), S(O)₂(C₁₋₆ alkyl) or NHC(O)heteroaryl}, aryl {substituted by nitrogen-containing heterocyclyl(C₁₋₄ alkyl), amino(C₁₋₄ alkyl), amino(C₁₋₄ alkoxy) or C₁₋₄ alkylamino(C₁₋₄ alkoxy) (itself optionally substituted by phenyl)}, heteroaryl {substituted by nitrogen-containing heterocyclyl(C₁₋₄ alkyl), amino(C₁₋₄ alkyl), amino(C₁₋₄ alkoxy) or C₁₋₄ alkylamino(C₁₋₄ alkoxy) (itself optionally substituted by phenyl)}, nitrogen-containing heterocyclyl {substituted by amino, aryl(C₁₋₄ alkyl) or heteroaryl(C₁₋₄ alkyl)}, aryl(C₁₋₄ alkyl) {substituted by amino(C₁₋₄ alkyl)} or C₃₋₇ cycloalkyl {substituted by nitrogen-containing heterocyclyl or amino};
provided that when R² is aryl or heteroaryl, each of which is optionally substituted by halogen, cyano, hydroxy, C₁₋₄ alkyl, C₁₋₄ alkoxy, CF₃, OCF₃, C₁₋₄ alkylthio, S(O)(C₁₋₄ alkyl), S(O)₂(C₁₋₄ alkyl) or C(O)₂(C₁₋₄ alkyl); then R¹ is not C₁₋₆ alkyl substituted by nitrogen-containing heterocyclyl;

### OR

L is CH and J is N(CH₂)ₘR⁹⁹; m is 1, 2, 3 or 4; R⁹⁹ is NH₂, phenyl or heteroaryl; AND
R¹ is C₁₋₆ alkyl {optionally substituted by hydroxyl, C₁₋₆ alkoxy, NR⁷⁷R⁸⁸, heterocyclyl (optionally substituted by oxo, hydroxy, C₁₋₆ alkyl, aryl, heteroaryl, aryl(C₁₋₄ alkyl), heterocyclyl or C(O)(C₁₋₄ alkyl)phenyl), aryl, heteroaryl, C₃₋₇ cycloalkyl, C₃₋₇ cycloalkyl(C₁₋₄ alkyl), CO₂H, CO₂(C₁₋₆ alkyl), aryl(C₁₋₄ alkoxy), aryl(C₁₋₄ alkylthio), S(O)₂(C₁₋₆ alkyl), NHC(O)heteroaryl or NHC(O)R⁶⁶}, C₁₋₆ alkoxy, C₃₋₆ cycloalkyl (optionally substituted by hydroxyl or C₁₋₆ alkyl), heterocyclyl {optionally substituted by oxo, hydroxy, C₁₋₆ alkyl, amino, aryl, heteroaryl, aryl(C₁₋₄ alkyl), heteroaryl(C₁₋₄ alkyl), heterocyclyl or C(O)(C₁₋₄ alkyl)phenyl}, aryl(C₁₋₄ alkyl) {substituted by amino(C₁₋₄ alkyl)}, aryl or heteroaryl;
   R⁶⁶ is C₁₋₆ alkyl or phenyl;
   R³, R⁴, R⁷⁷ and R³⁸ are, independently, hydrogen, C₁₋₆ alkyl or phenyl(C₁₋₄ alkyl); in addition to any substituents that might be specified above the foregoing nitrogen-containing heterocyclyl rings are optionally substituted by oxo, hydroxy, C₁₋₆ alkyl (itself optionally substituted by NH₂, NH(C₁₋₄ alkyl) or N(C₁₋₄ alkyl)₂), NH₂, aryl, heteroaryl, aryl(C₁₋₄ alkyl), heteroaryl(C₁₋₄ alkyl), heterocyclyl or C(O)(C₁₋₄ alkyl)phenyl; in addition to any required substituents that might be specified above the foregoing phenyl, aryl and heteroaryl moieties are, independently, optionally substituted by: halogen, cyano, nitro, hydroxy, S(O)_{q}R²⁴, OC(O)NR⁵R⁶, NR⁷R⁸, NR⁹C(O)R¹⁰, NR¹¹C(O)NR¹²R¹³, S(O)₂NR¹⁴R¹⁵, NR¹⁶S(O)₂R¹⁷, C(O)NR¹⁸R¹⁹, C(O)R²⁰, CO₂R²¹, NR²²CO₂R²³, C₁₋₆ alkyl, C₁₋₆ hydroxyalkyl, C₁₋₆ haloalkyl, C₁₋₆ alkoxy(C₁₋₆)alkyl, di(C₁₋₆)alkylamino(C₁₋₆)alkyl, C₁₋₆ alkoxy, C₁₋₆ haloalkoxy, C₁₋₆ alkoxy(C₁₋₆)alkoxy, C₁₋₆ alkylthio, C₂₋₆ alkenyl, C₂₋₆ alkynyl, C₃₋₁₀ cycloalkyl (itself optionally substituted by C₁₋₄ alkyl or oxo), methylenedioxy, difluoromethylenedioxy, phenyl, phenyl(C₁₋₄)alkyl, phenoxy, phenylthio, phenyl(C₁₋₄)alkoxy, heteroaryl, heteroaryl(C₁₋₄)alkyl, heteroaryloxy or heteroaryl(C₁₋₄)alkoxy; wherein any of the immediately foregoing phenyl and heteroaryl moieties are optionally substituted with halogen, hydroxy, nitro, S(O)ᵣ(C₁₋₄ alkyl), S(O)₂NH₂, S(O)₂NH(C₁₋₄ alkyl), S(O)₂N(C₁₋₄ alkyl)₂, cyano, C₁₋₄ alkyl, C₁₋₄ alkoxy, C(O)NH₂, C(O)NH(C₁₋₄ alkyl), C(O)N(C₁₋₄ alkyl)₂, CO₂H, CO₂(C₁₋₄ alkyl), NHC(O)(C₁₋₄ alkyl), NHS(O)₂(C₁₋₄ alkyl), C(O)(C₁₋₄ alkyl), CF₃ or OCF₃;
   A¹, E¹ and G¹ are, independently, hydrogen, halogen, cyano, hydroxy, C₁₋₄ alkyl, C₁₋₄ alkoxy, CF₃ or OCF₃;
   q and r are, independently, 0, 1 or 2;
   R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵, R²⁶, R²⁷, R², R²⁹, R³⁰ and R³¹ are, independently, C₁₋₆ alkyl {optionally substituted by halogen, hydroxy or C₁₋₆ alkoxy}, CH₂(C₂₋₆ alkenyl), phenyl {itself optionally substituted by halogen, hydroxy, nitro, NH₂, NH(C₁₋₄ alkyl), N(C₁₋₄ alkyl)₂, S(O)₂(C₁₋₄ alkyl), S(O)₂NH₂, S(O)₂NH(C₁₋₄ alkyl), S(O)₂N(C₁₋₄ alkyl)₂, cyano, C₁₋₄ alkyl, C₁₋₄ alkoxy, C(O)NH₂, C(O)NH(C₁₋₄ alkyl), C(O)N(C₁₋₄ alkyl)₂, CO₂H, CO₂(C₁₋₄ alkyl), NHC(O)(C₁₋₄ alkyl), NHS(O)₂(C₁₋₄ alkyl), C(O)(C₁₋₄ alkyl), CF₃ or OCF₃} or heteroaryl {itself optionally substituted by halogen, hydroxy, nitro, NH₂, NH(C₁₋₄ alkyl), N(C₁₋₄ alkyl)₂, S(O)₂(C₁₋₄ alkyl), S(O)₂NH₂, S(O)₂NH(C₁₋₄ alkyl), S(O)₂N(C₁₋₄ alkyl)₂, cyano, C₁₋₄ alkyl, C₁₋₄ alkoxy, C(O)NH₂, C(O)NH(C₁₋₄ alkyl), C(O)N(C₁₋₄ alkyl)₂, CO₂H, CO₂(C₁₋₄ alkyl), NHC(O)(C₁₋₄ alkyl), NHS(O)₂(C₁₋₄ alkyl), C(O)(C₁₋₄ alkyl), CF₃ or OCF₃};
   R⁵, R⁶, R⁷, R⁸, R⁹, R¹⁰, R¹¹, R¹², R¹³, R¹⁴, R¹⁵, R¹⁶, R¹⁸, R¹⁹, R²⁰, R²¹, R²², R²³, R²⁴, R²⁵, R²⁶, R²⁷, R²³, R²⁹ and R³⁰ can also be hydrogen;
   or a N-oxide thereof, or a pharmaceutically acceptable salt thereof.

Certain compounds of the present invention can exist in different isomeric forms (such as enantiomers, diastereomers, geometric isomers or tautomers). The present invention covers all such isomers and mixtures thereof in all proportions.

A pharmaceutically acceptable salt of a compound of formula (I) includes a salt prepared from a pharmaceutically acceptable non-toxic base, such as an inorganic or organic base. A salt derived from an inorganic base is, for example, an aluminium, calcium, potassium, magnesium, sodium or zinc salt. A salt derived from an organic base is, for example, a salt of a primary, secondary or tertiary amine, such as arginine, betaine, benzathine, caffeine, choline, chloroprocaine, cycloprocaine, N′,N′-dibenzylethylenediamine, diethanolamine, diethylamine, 2-diethyl-aminoethanol, 2-dimethylaminoethanol, ethanolamine, ethylendiamine, N-ethyl-morpholine, N-ethyl piperidine, glucamine, glucosamine, histidine, hydrabamine, isopropylamine, lysine, meglumine, morpholine, piperazine, piperidine, polyamine resins, procaine, purines, tertiary butylamine, theobromine, triethylamine, trimethylamine, tripropylamine, tromethamine or thanolamine.

A pharmaceutically acceptable salt of a compound of formula (I) also includes a quaternary ammonium salt, for example where an amine group in a compound of formula (I) reacts with a C₁₋₁₀ alkyl halide (for example a chloride, bromide or iodide) to form a quaternary ammonium salt.

A pharmaceutically acceptable salt also includes a salt of pharmaceutically acceptable organic acid, such as a carboxylic or sulphonic acid, for example: an acetate, adipate, alginate, ascorbate, aspartate, benzenesulphonate (besylate), benzoate, butyrate, camphorate, camphorsulphonate, camsylate, citrate, p-chlorobenzenesulphonate, cyclopentate, 2,5-dichlorobesyalte, digluconate, edisylate (ethane-1,2-disulfonate or ethane-1-(sulfonic acid)-2-sulfonate), esylate, ethanesulphonate, fumarate, formate, 2-furoate, 3-furoate, gluconate, glucoheptanate, glutamate, glutarate, glycerophosphate, glycolate, heptanoate, hexanoate, hippurate, 2-hydroxyethane sulfonate, lactate, lactobionate, laurate, malate, maleate, malonate, mandelate, methanesulphonate, 2-naphthalenesulfonate, napadisylate (naphthalene-1,5-disulfonate or naphthalene-1-(sulfonic acid)-5-sulfonate), nicotinate, oleate, orotate, oxalate, pantothenate, pamoate, pamoic, pectinate, 3-phenylpropionate, pivalate, propionate, pivolate, pyruvate, saccharinate, salicylate, stearate, succinate, tartrate, p-toluenesulphonate, transcinnamic acid, trifluoroacetate, xinafoate, xinofolate, xylate (p-xylene-2-sulphonic acid), undecanoate, 2-mesitylenesulphonate, 2-naphthalenesulphonate, D-mandelate, L-mandelate, 2,5-dichlorobenzenesulphonate, cinnamate or benzoate; or a salt of an inorganic acid such as a hydrobromide, hydrochloride, hydroiodide, sulphate, bisulfate, phosphate, nitrate, hemisulfate, thiocyanate, persulfate, phosphate or sulphonate salt. In another aspect of the invention the stoichiometry of the salt is, for example, a hemi-salt, or a mono- or di-salt.

A pharmaceutically acceptable salt of a compound of formula (I) can be prepared in situ during the final isolation and purification of a compound, or by separately reacting the compound or N-oxide with a suitable organic or inorganic acid and isolating the salt thus formed.

In one aspect of the invention acid addition salts are, for example, a hydrochloride, dihydrochloride, hydrobromide, phosphate, sulfate, acetate, diacetate, fumarate, maleate, malonate, succinate, tartrate, citrate, oxalate, methanesulfonate orp-toluenesulfonate. An alternative acid addition salt is a trifluoroacetate salt.

Alternatively, a suitable salt can be a quaternary ammonium salt formed by the reaction of a primary, secondary or tertiary amine group in a compound of formula (I) with, for example, a C₁₋₆ alkyl halide (such as methyl iodide or methyl bromide).

The compounds of the invention may exist as solvates (such as hydrates) and the present invention covers all such solvates.

Halogen includes fluorine, chlorine, bromine and iodine. Halogen is, for example, fluorine or chlorine.

Alkyl moieties are straight or branched chain and are, for example, methyl, ethyl, n-propyl, iso-propyl or tert-butyl. Haloalkyl is, for example C₂F₅, CF₃ or CHF₂. Alkoxy is, for example, methoxy or ethoxy; and haloalkoxy is, for example OCF₃ or OCHF₂.

Alkenyl is, for example, vinyl or prop-2-enyl. Alkynyl is, for example, propargyl.

Cycloalkyl is a mono- or bi-cyclic ring system which is saturated or unsaturated but not aromatic, and can, optionally, be fused to a benzene ring. It is, for example, cyclopropyl, cyclobutyl, cyclopentyl, cyclohexyl, indanyl or bicyclo[3.1.1]heptenyl. When cycloalkyl is substituted by nitrogen-containing heterocyclyl then the two rings can be joined in spiro-fashion (that is, one carbon is in both rings). C₃₋₇ Cycloalkyl(C₁₋₄ alkyl) is, for example, cyclopentylCH₂. Cycloalkyloxy is, for example, cyclopropyloxy, cyclopentyloxy or cyclohexyloxy. Cycloalkylalkoxy is, for example, (cyclopropyl)methoxy or 2-(cyclopropyl)ethoxy.

Nitrogen-containing heteocyclyl is a non-aromatic 5- or 6-membered ring (optionally fused to a benzene ring), comprising at least one nitrogen atom heteroatom and optionally a further heteroatom selected from the group comprising nitrogen, oxygen and sulphur. Nitrogen-containing heterocyclyl is, for example, azetidinyl, pyrrolidinyl, piperidinyl, piperazinyl, isoindolyl, morpholinyl, 3,8-diazabicyclo[3.2.1]octyl, 8-azabicyclo[2.2.2]octyl, 2-oxa-6-azabicyclo[5.4.0]undeca-7,9,11-trienyl, 7-oxa-10-azabicyclo[4.4.0]deca-1,3,5-trienyl or 6-thia-1,4-diazabicyclo[3.3.0]octa-4,7-dienyl.

When nitrogen-containing heterocyclyl is substituted by nitrogen-containing heterocyclyl or heterocyclyl then the two rings can be joined in spiro-fashion (that is, one carbon is in both rings).

Heterocyclyl is a non-aromatic 5- or 6-membered ring optionally fused to one or more other non-aromatic rings and optionally fused to a benzene ring, comprising at least one heteroatom selected from the group comprising nitrogen, oxygen and sulphur. Heterocyclyl is, for example, azetidinyl, pyrrolidinyl, piperidinyl, piperazinyl, isoindolyl, morpholinyl, 3,8-diazabicyclo[3.2.1]octyl, 8-azabicyclo[2.2.2]octyl, 2-oxa-6-azabicyclo[5.4.0]undeca-7,9,11-trienyl, 7-oxa-10-azabicyclo[4.4.0]deca-1,3,5-trienyl, 6-thia-1,4-diazabicyclo[3.3.0]octa-4,7-dienyl, tetrahydropyranyl, azabicyclo[3.2.1]octyl, 1,2,3,4-tetrahydroquinolinyl, 1,4-diazepinyl, quinuclidinyl, 9-oxa-2,8-diazaspiro[4.4]non-7-enyl, 1,2-dihydroquinazolinyl, 2,4,10-triazabicyclo[4.4.0]deca-1,3,5,8-tetraenyl or 2-oxa-5-aza-bicyclo[4.4.0]deca-7,9,11-trienyl.

Hydroxyalkyl is, for example, CH₂OH; C₁₋₆ alkoxy(C₁₋₆)alkyl is, for example CH₃OCH₂; and, C₁₋₆ alkoxy(C₁₋₆)alkoxy is, for example, CH₃OCH₂O. Dialkylaminoalkyl is, for example (CH₃)₂NCH₂ or (CH₃)(CH₃CH₂)NCH₂. Amino(C₁₋₄ alkyl) is, for example, CH₂NH₂. Amino(C₁₋₄ alkoxy) is, for example, OCH₂NH₂. C₁₋₄ Alkylamino(C₁₋₄ alkoxy) is, for example, CH₃NHCH₂O.

Aryl is, for example, phenyl or naphthyl. In one aspect aryl is phenyl. Aryl(C₁₋₄ alkyl) is, for example, benzyl. Aryl(C₁₋₄ alkoxy) is, for example, phenylmethoxy. Aryl(C 4 alkylthio) is, for example, phenylCH₂S.

Heteroaryl is, for example, an aromatic 5- or 6-membered ring, optionally fused to one or more other rings, comprising at least one heteroatom selected from the group comprising nitrogen, oxygen and sulphur; or an N-oxide thereof, or an S-oxide or S-dioxide thereof. Heteroaryl is, for example, furyl, thienyl (also known as thiophenyl), pyrrolyl, thiazolyl, isothiazolyl, pyrazolyl, oxazolyl, isoxazolyl, imidazolyl, [1,2,3]-thiadiazolyl, [1,2,4]-triazolyl, [1,2,3]-triazolyl, pyridinyl, pyrimidinyl, pyrazinyl, pyridazinyl, indolyl, benzo[b]furyl (also known as benzfuryl), benz[b]thienyl (also known as benzthienyl or benzthiophenyl), indazolyl, benzimidazolyl, 1,2,3-benztriazolyl, benzoxazolyl, 1,3-benzthiazolyl, 1,2,3-benzothiadiazolyl, an imidazopyridinyl (such as imidazo[1,2a]pyridinyl), thieno[3,2-b]pyridin-6-yl, 1,2,3-benzoxadiazolyl (also known as benzo[1,2,3]thiadiazolyl), 2,1,3-benzothiadiazolyl, benzofurazan (also known as 2,1,3-benzoxadiazolyl), quinoxalinyl, a pyrazolopyridine (for example 1H-pyrazolo[3,4-b]pyridinyl or pyrazolo[1,5-a]pyridinyl), an imidazopyridine (for example imidazo[1,2-a]pyridinyl or 5,6,7,8-tetrahydroimidazo[1,2-a]pyridinyl), a dihydropyrido[2,3-d]pyrimidine (for example 1,4-dihydropyrido[2,3-d]pyrimidinyl), quinolinyl, isoquinolinyl, a naphthyridinyl (for example [1,6]naphthyridinyl, [1,7]naphthyridinyl or [1,8]naphthyridinyl), 1,2,3-thiadiazolyl, 1H-pyrrolo[2,3-b]pyridinyl, thieno[2,3-b]pyridinyl, thieno[2,3-b]pyrazinyl, [1,2,4]triazolo[1,5-a]pyrimidinyl or 6,7-dihydro-5H-[1,3]thiazolo[3,2-a]pyrimidinyl; or an N-oxide thereof, or an S-oxide or S-dioxide thereof. A further example of heteroaryl is isoindolyl.

NHC(O)Heteroaryl is, for example, NHC(O)pyridinyl. Heteroaryl(C₁₋₄ alkyl) is, for example, pyridinylCH₂.

In one particular aspect the present invention provides a compound of formula (I) wherein in addition to any substituents that might be specified the nitrogen-containing heterocyclyl rings are optionally substituted by oxo, hydroxy, C₁₋₆ alkyl (itself optionally substituted by NH₂, NH(C₁₋₄ alkyl) or N(C₁₋₄ alkyl)₂), aryl, heteroaryl, aryl(C₁₋₄ alkyl), heteroaryl(C₁₋₄ alkyl), heterocyclyl or C(O)(C₁₋₄ alkyl)phenyl;

In another aspect the present invention provides a compound of formula (I) wherein E is CE¹. For example E is CF.

In a further aspect A is CA¹. For example A is CH.

In another aspect G¹ is hydrogen.

In a still further aspect A¹, E¹ and G¹ are, independently, hydrogen or halogen (for example fluoro).

In yet another aspect the present invention provides a compound of formula (I) wherein n and p are both 1.

In a further aspect the present invention provides a compound of formula (I) wherein L is CH.

In another aspect the present invention provides a compound of formula (I) wherein T is C(O).

In a still further aspect the present invention provides a compound of formula (I) wherein Y and W are both CH₂, L is CH, J is NH and T is C(O).

In another aspect the present invention provides a compound of formula (I) wherein Y and W are both CH₂, L is CH, J is N(CH₂)ₘ, m is 1, 2 or 3 (for example m is 2), T is C(O), and R¹ is heteroaryl optionally substituted as recited above (for example optionally substituted by halogen or amino(C₁₋₄ alkyl)).

In another aspect the present invention provides a compound of formula (I) wherein L is CH, J is NH; and R¹ is C₁₋₆ alkyl {substituted by either NR³R⁴ or nitrogen-containing heterocyclyl; and optionally additionally substituted by aryl, heteroaryl, C₃₋₇ cycloalkyl, C₃₋₇ cycloalkyl(C₁₋₄ alkyl), aryl(C₁₋₄ alkoxy), aryl(C₁₋₄ alkylthio), S(O)₂(C₁₋₆ alkyl) or NHC(O)heteroaryl}, aryl {substituted by nitrogen-containing heterocyclyl(C₁₋₄ alkyl), amino(C₁₋₄ alkyl), amino(C₁₋₄ alkoxy) or C₁₋₄ alkylamino(C₁₋₄ alkoxy) (itself optionally substituted by phenyl)}, heteroaryl {substituted by nitrogen-containing heterocyclyl(C₁₋₄ alkyl), amino(C₁₋₄ alkyl), amino(C₁₋₄ alkoxy) or C₁₋₄ alkylamino(C₁₋₄ alkoxy) (itself optionally substituted by phenyl)}, nitrogen-containing heterocyclyl {substituted by amino, aryl(C₁₋₁₄ alkyl) or heteroaryl(C₁₋₄ alkyl)}, aryl(C₁₋₁₄ alkyl) {substituted by amino(C₁₋₄ alkyl)} or C₃₋₇ cycloalkyl {substituted by nitrogen-containing heterocyclyl or amino}; nitrogen-containing heterocyclyl is optionally substituted by C₁₋₄ alkyl, NH₂, oxo, hydroxy, aryl, heteroaryl, aryl(C₁₋₄ alkyl) or C(O)(C₁₋₄ alkyl)phenyl; R³ and R⁴ are as defined above; provided that when R² is aryl or heteroaryl, each of which is optionally substituted by halogen, cyano, hydroxy, C₁₋₄ alkyl, C₁₋₄ alkoxy, CF₃, OCF₃, C₁₋₄ alkylthio, S(O)(C₁₋₄ alkyl), S(O)₂(C₁₋₄ alkyl) or C(O)₂(C₁₋₄ alkyl); then R¹ is not C₁₋₆ alkyl substituted by nitrogen-containing heterocyclyl.

In another aspect the present invention provides a compound of formula (I) wherein L is CH, J is NH; and R¹ is C₁₋₆ alkyl {substituted by NR³R⁴; and optionally additionally substituted by aryl, heteroaryl, C₃₋₇ cycloalkyl, C₃₋₇ cycloalkyl(C₁₋₄ alkyl), aryl(C₁₋₄ alkoxy), aryl(C₁₋₁₄ alkylthio), S(O)₂(C₁₋₆ alkyl) or NHC(O)heteroaryl}; wherein R³ and R⁴ are as defined above.

In a yet another aspect the present invention provides a compound of formula (I) wherein R³ and R⁴ are, independently, hydrogen or C₁₋₆ alkyl.

In a further aspect the present invention provides a compound of formula (I) wherein L is CH and J is N(CH₂)ₘNH₂; m is 1, 2, 3 or 4 (for example m is 2).

In a still further aspect the present invention provides a compound of formula (I) wherein L is CH and J is N(CH₂)ₘR⁹⁹; m is 1, 2, 3 or 4 (for example m is 1); and R⁹⁹ is phenyl (optionally substituted as described above).

In another aspect the present invention provides a compound of formula (I) wherein E¹ and G¹ are, independently, hydrogen or halogen (for example fluoro).

In yet another aspect the present invention provides a compound of formula (I) wherein n is 0.

In a further aspect the present invention provides a compound of formula (I) wherein n is 1.

In another aspect the present invention provides a compound of formula (I) wherein R² is tetrahydrothiopyranyl.

In a still further aspect the present invention provides a compound of formula (I) wherein R¹ is C₁₋₆ alkyl {substituted by NR³R⁴ or nitrogen-containing heterocyclyl} or heteroaryl {substituted by amino(C₁₋₄ alkyl)}; nitrogen-containing heterocyclyl being optionally substituted by phenyl(C₁₋₄ alkyl), C₁₋₄ alkyl or C(O)(C₁₋₄ alkyl)phenyl; and R³ and R⁴ are as defined above.

Compounds of the invention are described in the Examples. Each of the compounds of the Examples, or a pharmaceutically acceptable salt thereof, is a further aspect of the present invention.

The compounds of the present invention can be prepared as described below or by adapting methods known in the art. The compounds of the invention can be prepared as shown in the scheme below wherein T is C(O), and, R hydrogen (in which case the second step is omitted) or (CH₂)ₘR⁹⁹ (and if R⁹⁹ includes an NH₂ group then it is suitable protected and then deprotected at the end of the reaction sequence).

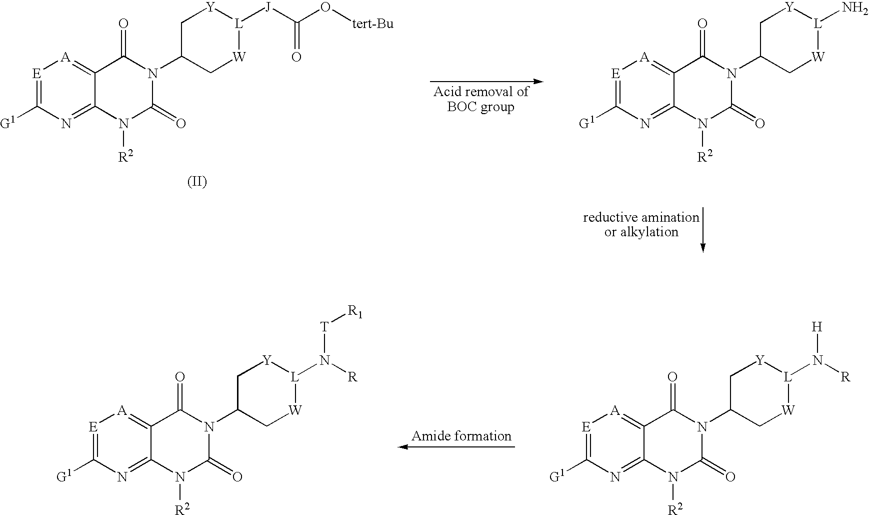

In a further aspect the invention provides a process for the preparation of a compound of formula (I), which comprises removing the Boc protecting group from a compound of formula (II)

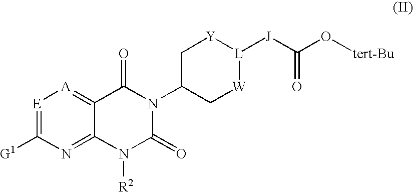

wherein m, A, R², G¹, E, Y, L, W, and J are as defined in formula (I), (for example with an acid such as trifluoroacetic acid or hydrochloric acid) and reacting the product so formed with a carboxylic acid of formula (III):

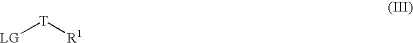

wherein R¹ and T are as defined in formula (I), and LG is a represents a leaving group (such as a halide). The process is carried out at a suitable temperature, generally between 0° C. and the boiling point of the solvent, in a suitable solvent such as dichloromethane or N-methylpyrrolidinone. The process is optionally carried out in the presence of a base and/or a coupling reagent such as HATU, HOAT, HOBT or DIEA. Suitable leaving groups LG include OH and halogen, particularly OH.

A compound of formula (II) wherein m, A, R², G¹, E, Y, L, W, and J are as defined in formula (I), can be prepared by condensing a compound of formula (IV):

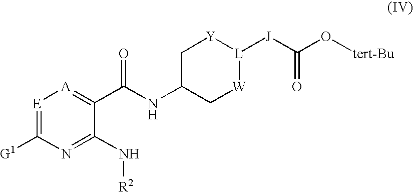

wherein m, A, G¹, E, Y, L, W, and J are as defined in formula (I), with a suitable carbonylating agent such as carbonyl diimidazole or ethyl chlorformate in the presence of a suitable base such as sodium hydride. The process is carried out at a suitable temperature, generally between 0° C. and the boiling point of the solvent, in a suitable solvent such as tetrahydrofuran.

A compound of formula (IV) wherein m, A, R², G¹, E, Y, L, W, T, and J are as defined in formula (I), can be prepared by reacting a compound of formula (V):

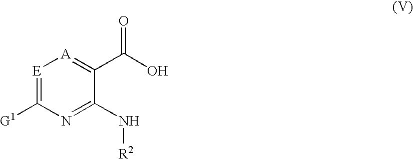

wherein A, R², m, G¹ and E are as defined in formula (I), with an amine of formula (VI)

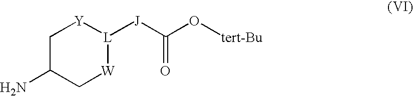

wherein Y, L, W, and J are as defined in formula (I). The process is carried out at a suitable temperature, generally between 0° C. and the boiling point of the solvent, in a suitable solvent such as dichloromethane. The process is optionally carried out in the presence of a base and a coupling reagent such as HATU, HOAT, HOBT or DIEA.

A compound of formula (V) wherein m, A, G¹ and E are as defined in formula (I), can be prepared by reacting a compound of formula (VII):

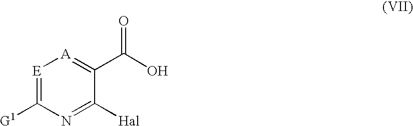

wherein A, G¹ and E are as defined in formula (I) and Hal represents a halogen atom, with R²—NH₂. The process is carried out at a suitable temperature, generally between 50° C. and the boiling point of the solvent, in a suitable solvent such as dimethylformamide. The process is optionally carried out in the presence of a base such as potassium carbonate.

The preparations of various intermediates are described in the literature or can be prepared by routine adaptation of methods described in the literature.

In the above processes it may be desirable or necessary to protect an acid group or a hydroxy or other potentially reactive group. Suitable protecting groups and details of processes for adding and removing such groups may be found in “Protective Groups in Organic Synthesis”, 3rd Edition (1999) by Greene and Wuts.

In another aspect the present invention provides processes for the preparation of compounds of formula (I).

The compounds of formula (I) have activity as pharmaceuticals, in particular as modulators of PDE 4 receptor activity, and may be used in the treatment of inflammatory diseases, asthma or COPD.

Examples of disease states that can be treated with a compound of the invention are:

1. respiratory tract: obstructive diseases of the airways including: asthma, including bronchial, allergic, intrinsic, extrinsic, exercise-induced, drug-induced (including aspirin and NSAID-induced) and dust-induced asthma, both intermittent and persistent and of all severities, and other causes of airway hyper-responsiveness; chronic obstructive pulmonary disease (COPD); bronchitis, including infectious and eosinophilic bronchitis; emphysema; bronchiectasis; cystic fibrosis; sarcoidosis; farmer's lung and related diseases; hypersensitivity pneumonitis; lung fibrosis, including cryptogenic fibrosing alveolitis, idiopathic interstitial pneumonias, fibrosis complicating anti-neoplastic therapy and chronic infection, including tuberculosis and aspergillosis and other fungal infections; complications of lung transplantation; vasculitic and thrombotic disorders of the lung vasculature, and pulmonary hypertension; antitussive activity including treatment of chronic cough associated with inflammatory and secretory conditions of the airways, and iatrogenic cough; acute and chronic rhinitis including rhinitis medicamentosa, and vasomotor rhinitis; perennial and seasonal allergic rhinitis including rhinitis nervosa (hay fever); nasal polyposis; acute viral infection including the common cold, and infection due to respiratory syncytial virus, influenza, coronavirus (including SARS) or adenovirus; or eosinophilic esophagitis;
2. bone and joints: arthritides associated with or including osteoarthritis/osteoarthrosis, both primary and secondary to, for example, congenital hip dysplasia; cervical and lumbar spondylitis, and low back and neck pain; osteoporosis; rheumatoid arthritis and Still's disease; seronegative spondyloarthropathies including ankylosing spondylitis, psoriatic arthritis, reactive arthritis and undifferentiated spondarthropathy; septic arthritis and other infection-related arthopathies and bone disorders such as tuberculosis, including Potts' disease and Poncet's syndrome; acute and chronic crystal-induced synovitis including urate gout, calcium pyrophosphate deposition disease, and calcium apatite related tendon, bursal and synovial inflammation; Behcet's disease; primary and secondary Sjogren's syndrome; systemic sclerosis and limited scleroderma; systemic lupus erythematosus, mixed connective tissue disease, and undifferentiated connective tissue disease; inflammatory myopathies including dermatomyositits and polymyositis; polymalgia rheumatica; juvenile arthritis including idiopathic inflammatory arthritides of whatever joint distribution and associated syndromes, and rheumatic fever and its systemic complications; vasculitides including giant cell arteritis, Takayasu's arteritis, Churg-Strauss syndrome, polyarteritis nodosa, microscopic polyarteritis, and vasculitides associated with viral infection, hypersensitivity reactions, cryoglobulins, and paraproteins; low back pain; Familial Mediterranean fever, Muckle-Wells syndrome, and Familial Hibernian Fever, Kikuchi disease; drug-induced arthalgias, tendonititides, and myopathies;
3. pain and connective tissue remodelling of musculoskeletal disorders due to injury [for example sports injury] or disease: arthritides (for example rheumatoid arthritis, osteoarthritis, gout or crystal arthropathy), other joint disease (such as intervertebral disc degeneration or temporomandibular joint degeneration), bone remodelling disease (such as osteoporosis, Paget's disease or osteonecrosis), polychondritits, scleroderma, mixed connective tissue disorder, spondyloarthropathies or periodontal disease (such as periodontitis);
4. skin: psoriasis, atopic dermatitis, contact dermatitis or other eczematous dermatoses, and delayed-type hypersensitivity reactions; phyto- and photodermatitis; seborrhoeic dermatitis, dermatitis herpetiformis, lichen planus, lichen sclerosus et atrophica, pyoderma gangrenosum, skin sarcoid, discoid lupus erythematosus, pemphigus, pemphigoid, epidermolysis bullosa, urticaria, angioedema, vasculitides, toxic erythemas, cutaneous eosinophilias, alopecia greata, male-pattern baldness, Sweet's syndrome, Weber-Christian syndrome, erythema multiforme; cellulitis, both infective and non-infective; panniculitis; cutaneous lymphomas, non-melanoma skin cancer and other dysplastic lesions; drug-induced disorders including fixed drug eruptions;
5. eyes: blepharitis; conjunctivitis, including perennial and vernal allergic conjunctivitis; iritis; anterior and posterior uveitis; choroiditis; autoimmune; degenerative or inflammatory disorders affecting the retina; ophthalmitis including sympathetic ophthalmitis; sarcoidosis; infections including viral, fungal, and bacterial;
6. gastrointestinal tract: glossitis, gingivitis, periodontitis; oesophagitis, including reflux; eosinophilic gastro-enteritis, mastocytosis, Crohn's disease, colitis including ulcerative colitis, proctitis, pruritis ani; coeliac disease, irritable bowel syndrome, and food-related allergies which may have effects remote from the gut (for example migraine, rhinitis or eczema);
7. abdominal: hepatitis, including autoimmune, alcoholic and viral; fibrosis and cirrhosis of the liver; cholecystitis; pancreatitis, both acute and chronic;
8. genitourinary: nephritis including interstitial and glomerulonephritis; nephrotic syndrome; cystitis including acute and chronic (interstitial) cystitis and Hunner's ulcer; acute and chronic urethritis, prostatitis, epididymitis, oophoritis and salpingitis; vulvo-vaginitis; Peyronie's disease; erectile dysfunction (both male and female);
9. allograft rejection: acute and chronic following, for example, transplantation of kidney, heart, liver, lung, bone marrow, skin or cornea or following blood transfusion; or chronic graft versus host disease;
10. CNS: Alzheimer's disease and other dementing disorders including CJD and nvCJD; amyloidosis; multiple sclerosis and other demyelinating syndromes; cerebral atherosclerosis and vasculitis; temporal arteritis; myasthenia gravis; acute and chronic pain (acute, intermittent or persistent, whether of central or peripheral origin) including visceral pain, headache, migraine, trigeminal neuralgia, atypical facial pain, joint and bone pain, pain arising from cancer and tumor invasion, neuropathic pain syndromes including diabetic, post-herpetic, and HIV-associated neuropathies; neurosarcoidosis; central and peripheral nervous system complications of malignant, infectious or autoimmune processes;
11. other auto-immune and allergic disorders including Hashimoto's thyroiditis, Graves' disease, Addison's disease, diabetes mellitus, idiopathic thrombocytopaenic purpura, eosinophilic fasciitis, hyper-IgE syndrome, antiphospholipid syndrome;
12. other disorders with an inflammatory or immunological component; including acquired immune deficiency syndrome (AIDS), leprosy, Sezary syndrome, and paraneoplastic syndromes;
13. cardiovascular: atherosclerosis, affecting the coronary and peripheral circulation; pericarditis; myocarditis, inflammatory and auto-immune cardiomyopathies including myocardial sarcoid; ischaemic reperfusion injuries; endocarditis, valvulitis, and aortitis including infective (for example syphilitic); vasculitides; disorders of the proximal and peripheral veins including phlebitis and thrombosis, including deep vein thrombosis and complications of varicose veins;
14. oncology: treatment of common cancers including prostate, breast, lung, ovarian, pancreatic, bowel and colon, stomach, skin and brain tumors and malignancies affecting the bone marrow (including the leukaemias) and lymphoproliferative systems, such as Hodgkin's and non-Hodgkin's lymphoma; including the prevention and treatment of metastatic disease and tumour recurrences, and paraneoplastic syndromes; or,
15. gastrointestinal tract: Coeliac disease, proctitis, eosinopilic gastro-enteritis, mastocytosis, Crohn's disease, ulcerative colitis, microscopic colitis, indeterminant colitis, irritable bowel disorder, irritable bowel syndrome, non-inflammatory diarrhea, food-related allergies which have effects remote from the gut, e.g., migraine, rhinitis and eczema.

According to a further feature of the present invention there is provided a method for treating a PDE 4 mediated disease state in a mammal, such as man, suffering from, or at risk of, said disease state, which comprises administering to a mammal in need of such treatment a therapeutically effective amount of a compound of the formula (I) or a pharmaceutically acceptable salt thereof.

The invention also provides a compound of the formula (I), or a pharmaceutically acceptable salt thereof, for use in therapy.

In another aspect the invention provides the use of a compound of formula (I), or a pharmaceutically acceptable salt thereof, in the manufacture of a medicament for use in therapy (for example modulating PDE 4 enzymatic activity).

The invention further provides the use of a compound of formula (I), or a pharmaceutically acceptable salt thereof, in the manufacture of a medicament for use in the treatment of:

1. respiratory tract: obstructive diseases of the airways including: asthma, including bronchial, allergic, intrinsic, extrinsic, exercise-induced, drug-induced (including aspirin and NSAID-induced) and dust-induced asthma, both intermittent and persistent and of all severities, and other causes of airway hyper-responsiveness; chronic obstructive pulmonary disease (COPD); bronchitis, including infectious and eosinophilic bronchitis; emphysema; bronchiectasis; cystic fibrosis; sarcoidosis; farmer's lung and related diseases; hypersensitivity pneumonitis; lung fibrosis, including cryptogenic fibrosing alveolitis, idiopathic interstitial pneumonias, fibrosis complicating anti-neoplastic therapy and chronic infection, including tuberculosis and aspergillosis and other fungal infections; complications of lung transplantation; vasculitic and thrombotic disorders of the lung vasculature, and pulmonary hypertension; antitussive activity including treatment of chronic cough associated with inflammatory and secretory conditions of the airways, and iatrogenic cough; acute and chronic rhinitis including rhinitis medicamentosa, and vasomotor rhinitis; perennial and seasonal allergic rhinitis including rhinitis nervosa (hay fever); nasal polyposis; acute viral infection including the common cold, and infection due to respiratory syncytial virus, influenza, coronavirus (including SARS) or adenovirus; or eosinophilic esophagitis;
2. bone and joints: arthritides associated with or including osteoarthritis/osteoarthrosis, both primary and secondary to, for example, congenital hip dysplasia; cervical and lumbar spondylitis, and low back and neck pain; osteoporosis; rheumatoid arthritis and Still's disease; seronegative spondyloarthropathies including ankylosing spondylitis, psoriatic arthritis, reactive arthritis and undifferentiated spondarthropathy; septic arthritis and other infection-related arthopathies and bone disorders such as tuberculosis, including Potts' disease and Poncet's syndrome; acute and chronic crystal-induced synovitis including urate gout, calcium pyrophosphate deposition disease, and calcium apatite related tendon, bursal and synovial inflammation; Behcet's disease; primary and secondary Sjogren's syndrome; systemic sclerosis and limited scleroderma; systemic lupus erythematosus, mixed connective tissue disease, and undifferentiated connective tissue disease; inflammatory myopathies including dermatomyositits and polymyositis; polymalgia rheumatica; juvenile arthritis including idiopathic inflammatory arthritides of whatever joint distribution and associated syndromes, and rheumatic fever and its systemic complications; vasculitides including giant cell arteritis, Takayasu's arteritis, Churg-Strauss syndrome, polyarteritis nodosa, microscopic polyarteritis, and vasculitides associated with viral infection, hypersensitivity reactions, cryoglobulins, and paraproteins; low back pain; Familial Mediterranean fever, Muckle-Wells syndrome, and Familial Hibernian Fever, Kikuchi disease; drug-induced arthalgias, tendonititides, and myopathies;
3. pain and connective tissue remodelling of musculoskeletal disorders due to injury [for example sports injury] or disease: arthritides (for example rheumatoid arthritis, osteoarthritis, gout or crystal arthropathy), other joint disease (such as intervertebral disc degeneration or temporomandibular joint degeneration), bone remodelling disease (such as osteoporosis, Paget's disease or osteonecrosis), polychondritits, scleroderma, mixed connective tissue disorder, spondyloarthropathies or periodontal disease (such as periodontitis);
4. skin: psoriasis, atopic dermatitis, contact dermatitis or other eczematous dermatoses, and delayed-type hypersensitivity reactions; phyto- and photodermatitis; seborrhoeic dermatitis, dermatitis herpetiformis, lichen planus, lichen sclerosus et atrophica, pyoderma gangrenosum, skin sarcoid, discoid lupus erythematosus, pemphigus, pemphigoid, epidermolysis bullosa, urticaria, angioedema, vasculitides, toxic erythemas, cutaneous eosinophilias, alopecia greata, male-pattern baldness, Sweet's syndrome, Weber-Christian syndrome, erythema multiforme; cellulitis, both infective and non-infective; panniculitis; cutaneous lymphomas, non-melanoma skin cancer and other dysplastic lesions; drug-induced disorders including fixed drug eruptions;
5. eyes: blepharitis; conjunctivitis, including perennial and vernal allergic conjunctivitis; iritis; anterior and posterior uveitis; choroiditis; autoimmune; degenerative or inflammatory disorders affecting the retina; ophthalmitis including sympathetic ophthalmitis; sarcoidosis; infections including viral, fungal, and bacterial;
6. gastrointestinal tract: glossitis, gingivitis, periodontitis; oesophagitis, including reflux; eosinophilic gastro-enteritis, mastocytosis, Crohn's disease, colitis including ulcerative colitis, proctitis, pruritis ani; coeliac disease, irritable bowel syndrome, and food-related allergies which may have effects remote from the gut (for example migraine, rhinitis or eczema);
7. abdominal: hepatitis, including autoimmune, alcoholic and viral; fibrosis and cirrhosis of the liver; cholecystitis; pancreatitis, both acute and chronic;
8. genitourinary: nephritis including interstitial and glomerulonephritis; nephrotic syndrome; cystitis including acute and chronic (interstitial) cystitis and Hunner's ulcer; acute and chronic urethritis, prostatitis, epididymitis, oophoritis and salpingitis; vulvo-vaginitis; Peyronie's disease; erectile dysfunction (both male and female);
9. allograft rejection: acute and chronic following, for example, transplantation of kidney, heart, liver, lung, bone marrow, skin or cornea or following blood transfusion; or chronic graft versus host disease;
10. CNS: Alzheimer's disease and other dementing disorders including CJD and nvCJD; amyloidosis; multiple sclerosis and other demyelinating syndromes; cerebral atherosclerosis and vasculitis; temporal arteritis; myasthenia gravis; acute and chronic pain (acute, intermittent or persistent, whether of central or peripheral origin) including visceral pain, headache, migraine, trigeminal neuralgia, atypical facial pain, joint and bone pain, pain arising from cancer and tumor invasion, neuropathic pain syndromes including diabetic, post-herpetic, and HIV-associated neuropathies; neurosarcoidosis; central and peripheral nervous system complications of malignant, infectious or autoimmune processes;
11. other auto-immune and allergic disorders including Hashimoto's thyroiditis, Graves' disease, Addison's disease, diabetes mellitus, idiopathic thrombocytopaenic purpura, eosinophilic fasciitis, hyper-IgE syndrome, antiphospholipid syndrome;
12. other disorders with an inflammatory or immunological component; including acquired immune deficiency syndrome (AIDS), leprosy, Sezary syndrome, and paraneoplastic syndromes;
13. cardiovascular: atherosclerosis, affecting the coronary and peripheral circulation; pericarditis; myocarditis, inflammatory and auto-immune cardiomyopathies including myocardial sarcoid; ischaemic reperfusion injuries; endocarditis, valvulitis, and aortitis including infective (for example syphilitic); vasculitides; disorders of the proximal and peripheral veins including phlebitis and thrombosis, including deep vein thrombosis and complications of varicose veins;
14. oncology: treatment of common cancers including prostate, breast, lung, ovarian, pancreatic, bowel and colon, stomach, skin and brain tumors and malignancies affecting the bone marrow (including the leukaemias) and lymphoproliferative systems, such as Hodgkin's and non-Hodgkin's lymphoma; including the prevention and treatment of metastatic disease and tumour recurrences, and paraneoplastic syndromes; or,
15. gastrointestinal tract: Coeliac disease, proctitis, eosinopilic gastro-enteritis, mastocytosis, Crohn's disease, ulcerative colitis, microscopic colitis, indeterminant colitis, irritable bowel disorder, irritable bowel syndrome, non-inflammatory diarrhea, food-related allergies which have effects remote from the gut, e.g., migraine, rhinitis and eczema;
in a mammal (for example man).

In a further aspect the invention provides a compound of formula (I), or a pharmaceutically acceptable salt thereof, for use in the treatment of asthma {such as bronchial, allergic, intrinsic, extrinsic or dust asthma, particularly chronic or inveterate asthma (for example late asthma or airways hyper-responsiveness)}; or COPD.

In a still further aspect a compound of formula (I), or a pharmaceutically acceptable salt thereof, is useful in the treatment of COPD.

The present invention also provides the use of a compound of formula (I), or a pharmaceutically acceptable salt thereof, in the manufacture of a medicament for use in the treatment of asthma {such as bronchial, allergic, intrinsic, extrinsic or dust asthma, particularly chronic or inveterate asthma (for example late asthma or airways hyper-responsiveness)}; or COPD.

In order to use a compound of the invention, or a pharmaceutically acceptable salt thereof, for the therapeutic treatment of a mammal, such as man, said ingredient is normally formulated in accordance with standard pharmaceutical practice as a pharmaceutical composition. Therefore in another aspect the present invention provides a pharmaceutical composition which comprises a compound of the formula (I), or a pharmaceutically acceptable salt thereof (active ingredient), and a pharmaceutically acceptable adjuvant, diluent or carrier.

In a further aspect the present invention provides a process for the preparation of said composition which comprises mixing active ingredient with a pharmaceutically acceptable adjuvant, diluent or carrier. Depending on the mode of administration, the pharmaceutical composition will, for example, comprise from 0.05 to 99% w (percent by weight), such as from 0.05 to 80% w, for example from 0.10 to 70% w, such as from 0.10 to 50% w, of active ingredient, all percentages by weight being based on total composition.

The pharmaceutical compositions of this invention may be administered in standard manner for the disease condition that it is desired to treat, for example by topical (such as to the lung and/or airways or to the skin), inhalation, oral, rectal or parenteral administration. For these purposes the compounds of this invention may be formulated by means known in the art. A suitable pharmaceutical composition of this invention is one suitable for oral administration in unit dosage form, for example a tablet or capsule which contains between 0.1 mg and 1 g of active ingredient.

Each patient may receive, for example, a dose of 0.001 mgkg⁻¹ to 100 mgkg⁻¹, for example in the range of 0.1 mgkg⁻¹ to 20 mgkg⁻¹, of the active ingredient administered, for example, 1 to 4 times per day.

The invention further relates to a combination therapy wherein a compound of the invention, or a pharmaceutically acceptable salt thereof, or a pharmaceutical composition or formulation comprising a compound of the invention, is administered concurrently or sequentially or as a combined preparation with another therapeutic agent or agents, for the treatment of one or more of the conditions listed.

In particular, for the treatment of the inflammatory diseases such as (but not restricted to) rheumatoid arthritis, osteoarthritis, asthma, allergic rhinitis, chronic obstructive pulmonary disease (COPD), psoriasis, and inflammatory bowel disease, the compounds of the invention may be combined with agents listed below.

Non-steroidal anti-inflammatory agents (hereinafter NSAIDs) including non-selective cyclo-oxygenase COX-1/COX-2 inhibitors whether applied topically or systemically (such as piroxicam, diclofenac, propionic acids such as naproxen, flurbiprofen, fenoprofen, ketoprofen and ibuprofen, fenamates such as mefenamic acid, indomethacin, sulindac, azapropazone, pyrazolones such as phenylbutazone, salicylates such as aspirin); selective COX-2 inhibitors (such as meloxicam, celecoxib, rofecoxib, valdecoxib, lumarocoxib, parecoxib and etoricoxib); cyclo-oxygenase inhibiting nitric oxide donors (CINODs); glucocorticosteroids (whether administered by topical, oral, intramuscular, intravenous, or intra-articular routes); methotrexate; leflunomide; hydroxychloroquine; d-penicillamine; auranofin or other parenteral or oral gold preparations; analgesics; diacerein; intra-articular therapies such as hyaluronic acid derivatives; and nutritional supplements such as glucosamine.

The present invention still further relates to the combination of a compound of the invention, or a pharmaceutically acceptable salt thereof, together with a cytokine or agonist or antagonist of cytokine function, (including agents which act on cytokine signalling pathways such as modulators of the SOCS system) including alpha-, beta-, and gamma-interferons; insulin-like growth factor type I (IGF-1); interleukins (IL) including IL1 to 17, and interleukin antagonists or inhibitors such as anakinra; tumour necrosis factor alpha (TNF-α) inhibitors such as anti-TNF monoclonal antibodies (for example infliximab; adalimumab, and CDP-870) and TNF receptor antagonists including immunoglobulin molecules (such as etanercept) and low-molecular-weight agents such as pentoxyfylline.

In addition the invention relates to a combination of a compound of the invention, or a pharmaceutically acceptable salt thereof, with a monoclonal antibody targeting B-Lymphocytes (such as CD20 (rituximab), MRA-aIL16R and T-Lymphocytes, CTLA4-Ig, HuMax Il-15).

The present invention still further relates to the combination of a compound of the invention, or a pharmaceutically acceptable salt thereof, with a modulator of chemokine receptor function such as an antagonist of CCR1, CCR2, CCR2A, CCR2B, CCR3, CCR4, CCR5, CCR6, CCR7, CCR8, CCR9, CCR10 and CCR11 (for the C—C family); CXCR1, CXCR2, CXCR3, CXCR4 and CXCR5 (for the C—X—C family) and CX₃CR1 for the C—X₃—C family.

The present invention further relates to the combination of a compound of the invention, or a pharmaceutically acceptable salt thereof, with an inhibitor of matrix metalloprotease (MMPs), i.e., the stromelysins, the collagenases, and the gelatinases, as well as aggrecanase; for example collagenase-1 (MMP-1), collagenase-2 (MMP-8), collagenase-3 (MMP-13), stromelysin-1 (MMP-3), stromelysin-2 (MMP-10), and stromelysin-3 (MMP-11) and MMP-9 and MMP-12, including agents such as doxycycline.

The present invention still further relates to the combination of a compound of the invention, or a pharmaceutically acceptable salt thereof, and a leukotriene biosynthesis inhibitor, 5-lipoxygenase (5-LO) inhibitor or 5-lipoxygenase activating protein (FLAP) antagonist such as; zileuton; ABT-761; fenleuton; tepoxalin; Abbott-79175; Abbott-85761; a N-(5-substituted)-thiophene-2-alkylsulfonamide; 2,6-di-tert-butylphenolhydrazones; a methoxytetrahydropyrans such as Zeneca ZD-2138; the compound SB-210661; a pyridinyl-substituted 2-cyanonaphthalene compound such as L-739,010; a 2-cyanoquinoline compound such as L-746,530; or an indole or quinoline compound such as MK-591, MK-886, and BAY×1005.

The present invention further relates to the combination of a compound of the invention, or a pharmaceutically acceptable salt thereof, and a receptor antagonist for leukotrienes (LT) B4, LTC4, LTD4, and LTE4. selected from the group consisting of the phenothiazin-3-yls such as L-651,392; amidino compounds such as CGS-25019c; benzoxalamines such as ontazolast; benzenecarboximidamides such as BIIL 284/260; and compounds such as zafirlukast, ablukast, montelukast, pranlukast, verlukast (MK-679), RG-12525, Ro-245913, iralukast (CGP 45715A), and BAY×7195.

The present invention still further relates to the combination of a compound of the invention, or a pharmaceutically acceptable salt thereof, and a phosphodiesterase (PDE) inhibitor such as a methylxanthanine including theophylline and aminophylline; a selective PDE isoenzyme inhibitor including a PDE4 inhibitor an inhibitor of the isoform PDE4D, or an inhibitor of PDE5.

The present invention further relates to the combination of a compound of the invention, or a pharmaceutically acceptable salt thereof, and a histamine type 1 receptor antagonist such as cetirizine, loratadine, desloratadine, fexofenadine, acrivastine, terfenadine, astemizole, azelastine, levocabastine, chlorpheniramine, promethazine, cyclizine, or mizolastine; applied orally, topically or parenterally.

The present invention still further relates to the combination of a compound of the invention, or a pharmaceutically acceptable salt thereof, and a proton pump inhibitor (such as omeprazole) or a gastroprotective histamine type 2 receptor antagonist.

The present invention further relates to the combination of a compound of the invention, or a pharmaceutically acceptable salt thereof, and an antagonist of the histamine type 4 receptor.

The present invention still further relates to the combination of a compound of the invention, or a pharmaceutically acceptable salt thereof, and an alpha-1/alpha-2 adrenoceptor agonist vasoconstrictor sympathomimetic agent, such as propylhexedrine, phenylephrine, phenylpropanolamine, ephedrine, pseudoephedrine, naphazoline hydrochloride, oxymetazoline hydrochloride, tetrahydrozoline hydrochloride, xylometazoline hydrochloride, tramazoline hydrochloride or ethylnorepinephrine hydrochloride.

The present invention further relates to the combination of a compound of the invention, or a pharmaceutically acceptable salt thereof, and an anticholinergic agent including muscarinic receptor (M1, M2, and M3) antagonist such as atropine, hyoscine, glycopyrrrolate, ipratropium bromide, tiotropium bromide, oxitropium bromide, pirenzepine or telenzepine.

The present invention still further relates to the combination of a compound of the invention, or a pharmaceutically acceptable salt thereof, and a beta-adrenoceptor agonist (including beta receptor subtypes 1-4) such as isoprenaline, salbutamol, formoterol, salmeterol, terbutaline, orciprenaline, bitolterol mesylate, or pirbuterol, or a chiral enantiomer thereof.

The present invention further relates to the combination of a compound of the invention, or a pharmaceutically acceptable salt thereof, and a chromone, such as sodium cromoglycate or nedocromil sodium.

The present invention still further relates to the combination of a compound of the invention, or a pharmaceutically acceptable salt thereof, with a glucocorticoid, such as flunisolide, triamcinolone acetonide, beclomethasone dipropionate, budesonide, fluticasone propionate, ciclesonide or mometasone furoate.

The present invention further relates to the combination of a compound of the invention, or a pharmaceutically acceptable salt thereof, with an agent that modulates a nuclear hormone receptor such as PPARs.

The present invention still further relates to the combination of a compound of the invention, or a pharmaceutically acceptable salt thereof, together with an immunoglobulin (Ig) or Ig preparation or an antagonist or antibody modulating Ig function such as anti-IgE (for example omalizumab).

The present invention further relates to the combination of a compound of the invention, or a pharmaceutically acceptable salt thereof, and another systemic or topically-applied anti-inflammatory agent, such as thalidomide or a derivative thereof, a retinoid, dithranol or calcipotriol.

The present invention still further relates to the combination of a compound of the invention, or a pharmaceutically acceptable salt thereof, and combinations of aminosalicylates and sulfapyridine such as sulfasalazine, mesalazine, balsalazide, and olsalazine; and immunomodulatory agents such as the thiopurines, and corticosteroids such as budesonide.

The present invention further relates to the combination of a compound of the invention, or a pharmaceutically acceptable salt thereof, together with an antibacterial agent such as a penicillin derivative, a tetracycline, a macrolide, a beta-lactam, a fluoroquinolone, metronidazole, an inhaled aminoglycoside; an antiviral agent including acyclovir, famciclovir, valaciclovir, ganciclovir, cidofovir, amantadine, rimantadine, ribavirin, zanamavir and oseltamavir; a protease inhibitor such as indinavir, nelfinavir, ritonavir, and saquinavir; a nucleoside reverse transcriptase inhibitor such as didanosine, lamivudine, stavudine, zalcitabine or zidovudine; or a non-nucleoside reverse transcriptase inhibitor such as nevirapine or efavirenz.

The present invention still further relates to the combination of a compound of the invention, or a pharmaceutically acceptable salt thereof, and a cardiovascular agent such as a calcium channel blocker, a beta-adrenoceptor blocker, an angiotensin-converting enzyme (ACE) inhibitor, an angiotensin-2 receptor antagonist; a lipid lowering agent such as a statin or a fibrate; a modulator of blood cell morphology such as pentoxyfylline; thrombolytic, or an anticoagulant such as a platelet aggregation inhibitor.

The present invention further relates to the combination of a compound of the invention, or a pharmaceutically acceptable salt thereof, and a CNS agent such as an antidepressant (such as sertraline), an anti-Parkinsonian drug (such as deprenyl, L-dopa, ropinirole, pramipexole, a MAOB inhibitor such as selegine and rasagiline, a comP inhibitor such as tasmar, an A-2 inhibitor, a dopamine reuptake inhibitor, an NMDA antagonist, a nicotine agonist, a dopamine agonist or an inhibitor of neuronal nitric oxide synthase), or an anti-Alzheimer's drug such as donepezil, rivastigmine, tacrine, a COX-2 inhibitor, propentofylline or metrifonate.

The present invention still further relates to the combination of a compound of the invention, or a pharmaceutically acceptable salt thereof, and an agent for the treatment of acute or chronic pain, such as a centrally or peripherally-acting analgesic (for example an opioid or derivative thereof), carbamazepine, phenyloin, sodium valproate, amitryptiline or other anti-depressant agents, paracetamol, or a non-steroidal anti-inflammatory agent.

The present invention further relates to the combination of a compound of the invention, or a pharmaceutically acceptable salt thereof, together with a parenterally or topically-applied (including inhaled) local anaesthetic agent such as lignocaine or a derivative thereof.

A compound of the present invention, or a pharmaceutically acceptable salt thereof, can also be used in combination with an anti-osteoporosis agent including a hormonal agent such as raloxifene, or a biphosphonate such as alendronate.

The present invention still further relates to the combination of a compound of the invention, or a pharmaceutically acceptable salt thereof, together with a: (i) tryptase inhibitor; (ii) platelet activating factor (PAF) antagonist; (iii) interleukin converting enzyme (ICE) inhibitor; (iv) IMPDH inhibitor; (v) adhesion molecule inhibitors including VLA-4 antagonist; (vi) cathepsin; (vii) kinase inhibitor such as an inhibitor of tyrosine kinase (such as Btk, Itk, Jak3 or MAP, for example Gefitinib or Imatinib mesylate), a serine/threonine kinase (such as an inhibitor of a MAP kinase such as p38, JNK, protein kinase A, B or C, or IKK), or a kinase involved in cell cycle regulation (such as a cylin dependent kinase); (viii) glucose-6 phosphate dehydrogenase inhibitor; (ix) kinin-B.sub1.- or B.sub2.-receptor antagonist; (x) anti-gout agent, for example colchicine; (xi) xanthine oxidase inhibitor, for example allopurinol; (xii) uricosuric agent, for example probenecid, sulfinpyrazone or benzbromarone; (xiii) growth hormone secretagogue; (xiv) transforming growth factor (TGFβ); (xv) platelet-derived growth factor (PDGF); (xvi) fibroblast growth factor for example basic fibroblast growth factor (bFGF); (xvii) granulocyte macrophage colony stimulating factor (GM-CSF); (xviii) capsaicin cream; (xix) tachykinin NK.sub 1. or NK.sub3. receptor antagonist such as NKP-608C, SB-233412 (talnetant) or D-4418; (xx) elastase inhibitor such as UT-77 or ZD-0892; (xxi) TNF-alpha converting enzyme inhibitor (TACE); (xxii) induced nitric oxide synthase (iNOS) inhibitor; (xxiii) chemoattractant receptor-homologous molecule expressed on TH2 cells, (such as a CRTH2 antagonist); (xxiv) inhibitor of p38; (xxv) agent modulating the function of Toll-like receptors (TLR), (xxvi) agent modulating the activity of purinergic receptors such as P2×7; (xxvii) inhibitor of transcription factor activation such as NFkB, API, or STATS; or (xxviii) a glucocorticoid receptor (GR-receptor) agonist.

In a further embodiment the present invention provides a pharmaceutical product comprising, in combination, a first active ingredient which is a compound of formula (I), or a pharmaceutically acceptable salt thereof, as hereinbefore described, and at least one further active ingredient selected from:—

a β2. adrenoceptor agonist,

a modulator of chemokine receptor function,

an inhibitor of kinase function,

a protease inhibitor,

a steroidal glucocorticoid receptor agonist,

an anticholinergic agent, and a

a non-steroidal glucocorticoid receptor agonist.

The pharmaceutical product according to this embodiment may, for example, be a pharmaceutical composition comprising the first and further active ingredients in admixture. Alternatively, the pharmaceutical product may, for example, comprise the first and further active ingredients in separate pharmaceutical preparations suitable for simultaneous, sequential or separate administration to a patient in need thereof. The pharmaceutical product of this embodiment is of particular use in treating respiratory diseases such as asthma, COPD or rhinitis.

Examples of a β₂-adrenoceptor agonist that may be used in the pharmaceutical product according to this embodiment include metaproterenol, isoproterenol, isoprenaline, albuterol, salbutamol (e.g. as sulphate), formoterol (e.g. as fumarate), salmeterol (e.g. as xinafoate), terbutaline, orciprenaline, bitolterol (e.g. as mesylate), pirbuterol or indacaterol. The β₂-adrenoceptor agonist of this embodiment may be a long-acting β₂-agonists, for example salmeterol (e.g. as xinafoate), formoterol (e.g. as fumarate), bambuterol (e.g. as hydrochloride), carmoterol (TA 2005, chemically identified as 2(1H)-Quinolone, 8-hydroxy-5-[1-hydroxy-2-[[2-(4-methoxy-phenyl)-1-methylethyl]-amino]ethyl]-monohydrochloride, [R—(R*,R*)] also identified by Chemical Abstract Service Registry Number 137888-11-0 and disclosed in U.S. Pat. No. 4,579,854), indacaterol (CAS no 312753-06-3; QAB-149), formanilide derivatives e.g. 3-(4-{[6-({(2R)-2-[3-(formylamino)-4-hydroxyphenyl]-2-hydroxyethyl}amino)hexyl]oxy}-butyl)-benzenesulfonamide as disclosed in WO 2002/76933, benzenesulfonamide derivatives e.g. 3-(4-{[6-({(2R)-2-hydroxy-2-[4-hydroxy-3-(hydroxy-methyl)phenyl]ethyl}amino)-hexyl]oxy}butyl)benzenesulfonamide as disclosed in WO 2002/88167, aryl aniline receptor agonists as disclosed in WO 2003/042164 and WO 2005/025555, indole derivatives as disclosed in WO 2004/032921 and US 2005/222144, and compounds GSK 159797, GSK 159802, GSK 597901, GSK 642444 and GSK 678007.

Examples of a modulator of chemokine receptor function that may be used in the pharmaceutical product according to this embodiment include a CCR1 receptor antagonist.

Examples of an inhibitor of kinase function that may be used in the pharmaceutical product according to this embodiment include a p38 kinase inhibitor and an IKK inhibitor.

Examples of a protease inhibitor that may be used in the pharmaceutical product according to this embodiment include an inhibitor of neutrophil elastase or an inhibitor of MMP12.

Examples of a steroidal glucocorticoid receptor agonist that may be used in the pharmaceutical product according to this embodiment include budesonide, fluticasone (e.g. as propionate ester), mometasone (e.g. as furoate ester), beclomethasone (e.g. as 17-propionate or 17,21-dipropionate esters), ciclesonide, loteprednol (as e.g. etabonate), etiprednol (as e.g. dicloacetate), triamcinolone (e.g. as acetonide), flunisolide, zoticasone, flumoxonide, rofleponide, butixocort (e.g. as propionate ester), prednisolone, prednisone, tipredane, steroid esters e.g. 6α,9α-difluoro-17α-[(2-furanylcarbonyl)oxy]-11β-hydroxy-16α-methyl-3-oxo-androsta-1,4-diene-17β-carbothioic acid S-fluoromethyl ester, 6α,9α-difluoro-11β-hydroxy-16α-methyl-3-oxo-17α-propionyloxy-androsta-1,4-diene-17β-carbothioic acid S-(2-oxo-tetrahydro-furan-3S-yl) ester and 6α,9α-difluoro-11β-hydroxy-16α-methyl-17α-[(4-methyl-1,3-thiazole-5-carbonyl)oxy]-3-oxo-androsta-1,4-diene-17β-carbothioic acid S-fluoromethyl ester, steroid esters according to DE 4129535, steroids according to WO 2002/00679, WO 2005/041980, or steroids GSK 870086, GSK 685698 and GSK 799943.

Examples of an anticholinergic agent that may be used in the pharmaceutical product according to this embodiment include for example a muscarinic receptor antagonist (for example a M1, M2 or M3 antagonist, such as a M3 antagonist) for example ipratropium (e.g. as bromide), tiotropium (e.g. as bromide), oxitropium (e.g. as bromide), tolterodine, pirenzepine, telenzepine, glycopyrronium bromide (such as R,R-glycopyrronium bromide or a mixture of R,S- and S,R-glycopyrronium bromide); mepensolate (e.g. as bromide), a quinuclidine derivative such as 3(R)-(2-hydroxy-2,2-dithien-2-ylacetoxy)-1-(3-phenoxypropyl)-1-azonia-bicyclo[2.2.2]octane bromide as disclosed in US 2003/0055080, quinuclidine derivatives as disclosed in WO 2003/087096 and WO 2005/115467 and DE 10050995; or GSK 656398 or GSK 961081.

Examples of a modulator of a non-steroidal glucocorticoid receptor agonist that may be used in the pharmaceutical product according to this embodiment include those described in WO2006/046916.

A compound of the invention, or a pharmaceutically acceptable salt thereof, can also be used in combination with an existing therapeutic agent for the treatment of cancer, for example suitable agents include:

(i) an antiproliferative/antineoplastic drug or a combination thereof, as used in medical oncology, such as an alkylating agent (for example cis-platin, carboplatin, cyclophosphamide, nitrogen mustard, melphalan, chlorambucil, busulphan or a nitrosourea); an antimetabolite (for example an antifolate such as a fluoropyrimidine like 5-fluorouracil or tegafur, raltitrexed, methotrexate, cytosine arabinoside, hydroxyurea, gemcitabine or paclitaxel); an antitumour antibiotic (for example an anthracycline such as adriamycin, bleomycin, doxorubicin, daunomycin, epirubicin, idarubicin, mitomycin-C, dactinomycin or mithramycin); an antimitotic agent (for example a vinca alkaloid such as vincristine, vinblastine, vindesine or vinorelbine, or a taxoid such as taxol or taxotere); or a topoisomerase inhibitor (for example an epipodophyllotoxin such as etoposide, teniposide, amsacrine, topotecan or a camptothecin);
(ii) a cytostatic agent such as an antioestrogen (for example tamoxifen, toremifene, raloxifene, droloxifene or iodoxyfene), an oestrogen receptor down regulator (for example fulvestrant), an antiandrogen (for example bicalutamide, flutamide, nilutamide or cyproterone acetate), a LHRH antagonist or LHRH agonist (for example goserelin, leuprorelin or buserelin), a progestogen (for example megestrol acetate), an aromatase inhibitor (for example as anastrozole, letrozole, vorazole or exemestane) or an inhibitor of 5α-reductase such as finasteride;
(iii) an agent which inhibits cancer cell invasion (for example a metalloproteinase inhibitor like marimastat or an inhibitor of urokinase plasminogen activator receptor function);
(iv) an inhibitor of growth factor function, for example: a growth factor antibody (for example the anti-erb b2 antibody trastuzumab, or the anti-erb b1 antibody cetuximab [C225]), a farnesyl transferase inhibitor, a tyrosine kinase inhibitor or a serine/threonine kinase inhibitor, an inhibitor of the epidermal growth factor family (for example an EGFR family tyrosine kinase inhibitor such as N-(3-chloro-4-fluorophenyl)-7-methoxy-6-(3-morpholinopropoxy)quinazolin-4-amine (gefitinib, AZD1839), N-(3-ethynylphenyl)-6,7-bis(2-methoxyethoxy)quinazolin-4-amine (erlotinib, OSI-774) or 6-acrylamido-N-(3-chloro-4-fluorophenyl)-7-(3-morpholinopropoxy)quinazolin-4-amine (CI 1033)), an inhibitor of the platelet-derived growth factor family, or an inhibitor of the hepatocyte growth factor family;
(v) an antiangiogenic agent such as one which inhibits the effects of vascular endothelial growth factor (for example the anti-vascular endothelial cell growth factor antibody bevacizumab, a compound disclosed in WO 97/22596, WO 97/30035, WO 97/32856 or WO 98/13354), or a compound that works by another mechanism (for example linomide, an inhibitor of integrin αvβ3 function or an angiostatin);
(vi) a vascular damaging agent such as combretastatin A4, or a compound disclosed in WO 99/02166, WO 00/40529, WO 00/41669, WO 01/92224, WO 02/04434 or WO 02/08213;
(vii) an agent used in antisense therapy, for example one directed to one of the targets listed above, such as ISIS 2503, an anti-ras antisense;
(viii) an agent used in a gene therapy approach, for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy; or,
(ix) an agent used in an immunotherapeutic approach, for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies.

The invention will now be illustrated by the following non-limiting examples in which, unless stated otherwise:

(i) when given, ¹H NMR data is quoted and is in the form of delta values for major diagnostic protons, given in parts per million (ppm) relative to tetramethylsilane (TMS) as an internal standard, determined at 300 MHz or 400 MHz using perdeuterio DMSO-D6 (CD₃SOCD₃) or CDCl₃ as the solvent unless otherwise stated;
(ii) mass spectra (MS) were run with an electron energy of 70 electron volts in the chemical ionisation (CI) mode using a direct exposure probe. Where indicated ionisation was effected by electrospray ionisation (ES), or atmospheric pressure chemical ionisation (APCI), or multimode ionisation, a combination of ES ionisation and APCI. Where values for m/z are given, generally only ions which indicate the parent mass are reported, and the mass ions quoted are the positive or negative mass ions: [M]⁺, [M+H]⁺ or [M−H]⁻;
(iii) the title and sub-title compounds of the examples and methods were named using the index name program from Advanced Chemistry Development Inc, version 8.00 or were named using the IUPAC name program from openeye and stereochemical descriptors added by hand. (See www.eyesopen.com/products/applications/ogham.html)
(iv) unless stated otherwise, reverse phase HPLC was conducted using a Symmetry™, NovaPak™ or Xterra™ reverse phase silica column, all available from Waters Corp.
(v) the following abbreviations are used:

| | |
|---|---|
| DMF | N,N-Dimethylformamide |
| NMP | 1-N-Methyl-2-pyrrolidinone |
| HOAT | 1-Hydroxy-7-azabenzotriazole |
| DIEA | N,N-Diisopropylethylamine |
| HATU | O-(7-Benzotriazol-1-yl)-N,N,N′,N′- |
| | tetramethyluronium hexafluorophosphate |
| THF | Tetrahydrofuran |
| DCM | Dichloromethane |
| BOC | tert-butoxycarbonyl |
| HPLC | High pressure liquid chromatography |
| d | Day(s) |
| h | Hour(s) |
| min | Minute(s) |

The starting materials for the Examples below are either commercially available or readily prepared by standard methods from known starting materials (The compounds are named using the index name program from Advanced Chemistry Development Inc, version 8.00)

### Preparation 1

### 5-Fluoro-2-(tetrahydro-2H-thiopyran-4-ylamino)nicotinic acid

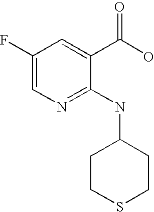

2-Chloro-5-fluoronicotinic acid (5.27 g, 30 mmol) and K₂CO₃ (5 g, 36 mmol) were added to dry DMF (30 ml) under an argon atmosphere. Copper (95 mg, 1.8 mmol), methanol-washed, dried copper(I) bromide (215 mg, 1.5 mmol) and tetrahydro-2H-thiopyran-4-amine (6 g, 51 mmol) were added and the mixture was stirred at 150° C. for four hours. Ethyl acetate was added and the crude product was washed twice with 0.5 M aqueous citric acid, the organic solvents dried over Na₂SO₄, filtered and the solvent removed in vacuo to afford the title compound (6 g, 78%).

¹H NMR (400 MHz, DMSO-d₆): δ 13.43 (1H, brs), 8.31 (1H, d), 7.97 (1H, brd), 7.91 (1H, dd), 3.99 (1H, brs), 2.76-2.61 (4H, m), 2.21 (2H, m), 1.64-1.53 (2H, m).

APCI-MS m/z: 257 [MH⁺].

### Preparation 2

### tert-Butyl [cis-4-({[5-fluoro-2-(tetrahydro-2H-thiopyran-4-ylamino)pyridin-3-yl]carbonyl}amino)cyclohexyl]carbamate

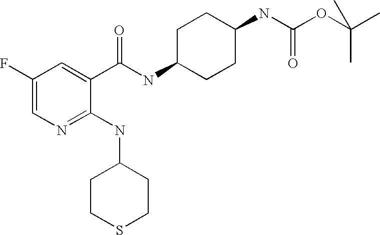

A mixture of 5-fluoro-2-(tetrahydro-2H-thiopyran-4-ylamino)nicotinic acid (5.9 g, 23 mmol), tert-butyl (cis-4-aminocyclohexyl)carbamate (5.42 g, 25.3 mmol), HATU (9.6 g, 25.3 mmol), HOAT (3.44 g, 25.3 mmol) and DIEA (12 ml, 70 mmol) in NMP (100 ml) was stirred for 10 min at room temperature (at pH 8-9 adjusted with DIEA). Ethyl acetate was added and the crude product was washed twice with 0.5 M aqueous citric acid, aqueous sodium hydrogencarbonate and water. The organic solvents were dried over Na₂SO₄, filtered and removed in vacuum. The residue was purified by flash chromatography on silica using ethyl acetate/heptane (1:3) as eluent to give the title compound (8.45 g, 81%).

¹H NMR (400 MHz, DMSO-d₆): δ 8.22 (1H, d), 8.17 (1H, brd), 8.16 (1H, brd), 7.96 (1H, dd), 6.61 (1H, brs), 3.92 (1H, brs), 3.77 (1H, brs), 3.40 (1H, brs), 2.74-2.60 (4H, m), 2.18 (2H, m), 1.70 (4H, m), 1.54 (6H, m), 1.39 (9H, s).

APCI-MS m/z: 453 [MH⁺].

### Preparation 3

### tert-Butyl {cis-4-[6-fluoro-2,4-dioxo-1-(tetrahydro-2H-thiopyran-4-yl)-1,4-dihydropyrido[2,3-d]pyrimidin-3(2H)-yl]cyclohexyl}carbamate

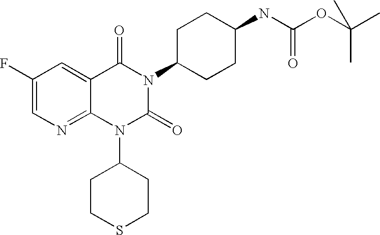

50% NaH in oil (1.24 g, 25.7 mmol) was added in 10 portions over a period of one hour to a solution of tert-butyl [cis-4-({[5-fluoro-2-(tetrahydro-2H-thiopyran-4-ylamino)pyridin-3-yl]carbonyl}amino)cyclohexyl]carbamate (3.9 g, 8.58 mmol) and 1,1′-carbonyldiimidazole (4.17 g, 25.7 mmol) in dry NMP (100 ml) under an argon atmosphere. The mixture was stirred at room temperature for two days. Ethyl acetate was added and the crude product was washed twice with 0.5 M aqueous citric acid, aqueous sodium hydrogencarbonate, and water. The organic solvents were dried over Na₂SO₄, filtered and removed in vacuum. The residue was purified by flash chromatography on silica using ethyl acetate/heptane (1:4) as eluent to give the title compound (1.95 mg, 48%) and 0.9 g (23%) of recovered starting material.

¹H NMR (400 MHz, DMSO-d₆): δ 8.78 (1H, d), 8.21 (1H, dd), 6.58 (1H, brs), 5.20 (1H, brs), 4.72 (1H, brt), 3.56 (1H, brs), 2.84-2.68 (6H, m), 2.58 (2H, q), 2.01-1.86 (4H, m), 1.49 (2H, brt), 1.42 (11H, brs).

APCI-MS m/z: 379 [MH⁺-tBOC].

### Preparation 4

### 3-(cis-4-Aminocyclohexyl)-6-fluoro-1-(tetrahydro-2H-thiopyran-4-yl)pyrido[2,3-d]pyrimidine-2,4(1H,3H)-dione hydrochloride

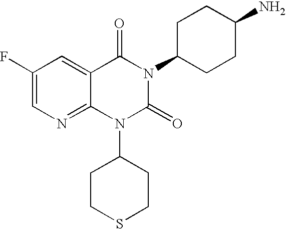

A mixture of tert-butyl {cis-4-[6-fluoro-2,4-dioxo-1-(tetrahydro-2H-thiopyran-4-yl)-1,4-dihydropyrido[2,3-d]pyrimidin-3(2H)-yl]cyclohexyl}carbamate (0.36 g, 0.75 mmol) and 4 M HCl in 1,4-Dioxane (10 ml) was stirred at room temperature for one hour. The solvents were removed and the pure crude product was used directly.

APCI-MS m/z: 379 [MH⁺].

### EXAMPLE 1

### N-(2-aminoethyl)-N-{cis-4-[6-fluoro-2,4-dioxo-1-(tetrahydro-2H-thiopyran-4-yl)-1,4-dihydropyrido[2,3-d]pyrimidin-3(2H)-yl]cyclohexyl}imidazo[1,2-a]pyridine-2-carboxamide

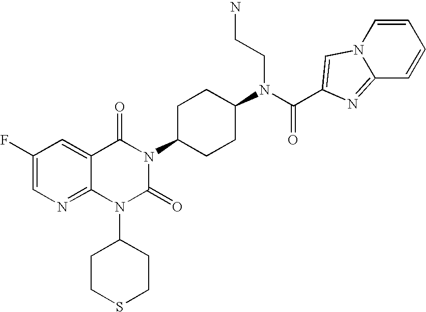

### Step (a) tert-butyl [2-({cis-4-[6-fluoro-2,4-dioxo-1-(tetrahydro-2H-thiopyran-4-yl)-1,4-dihydropyrido[2,3-d]pyrimidin-3(2H)-yl]cyclohexyl}amino)ethyl]carbamate

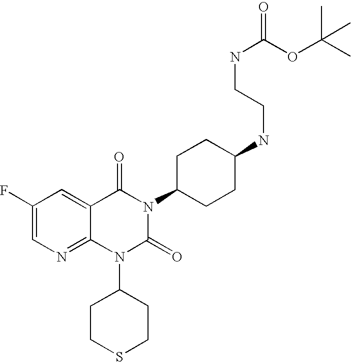

3-(4-Amino-cyclohexyl)-6-fluoro-1-(tetrahydro-thiopyran-4-yl)-1H-pyrido[2,3-d]pyrimidine-2,4-dione (250 mg, 0.7 mmol) was dissolved in 1,2-dichloroethane (10 ml) and tert-butyl-N-(2-oxoethyl)carbamate (95 mg, 0.6 mmol) was added, followed by sodium triacetoxyborohydride (126 mg, 0.6 mmol). The mixture was then stirred at room temperature overnight. The crude reaction was quenched by pouring into water, and the pH adjusted to 10 by the addition of 1M sodium hydroxide solution. The crude product was then purified by flash chromatography on silica using 2% methanol in ethyl acetate as the eluent to afford the sub-title compound (198 mg, 54%).

¹H NMR (400 MHz, CDCl₃) δ 8.48 (d, 1H), 8.13 (dd, 1H), 5.19 (s, 2H), 4.90 (m, 1H), 3.26 (d, 2H), 2.94 (m, 6H), 2.69 (m, 6H), 2.00 (m, 2H), 1.91 (m, 2H), 1.58 (m, 4H), 1.51 (s, 9H) APCI (Multimode) m/z: 522 [M+H]

### Step (b) N-(2-aminoethyl)-N-{cis-4-[6-fluoro-2,4-dioxo-1-(tetrahydro-2H-thiopyran-4-yl)-1,4-dihydropyrido[2,3-d]pyrimidin-3(2H)-yl]cyclohexyl}imidazo[1,2-a]pyridine-2-carboxamide

Imidazole[1,2a]pyridine-2-carboxylic acid (72 mg, 0.43 mmol) was dissolved in dry DMF (5 ml) and DIEA (0.2 ml, 1.15 mmol) was added, followed by HATU (164 mg, 0.43 mmol) and the mixture stirred for 10 min. (2-{4-[6-Fluoro-2,4-dioxo-1-(tetrahydro-thiopyran-4-yl)-1,4-dihydro-2H-pyrido[2,3-d]pyrimidin-3-yl]-cyclohexylamino}-ethyl)-carbamic acid tert-butyl ester (170 mg, 0.32 mmol) was added and the mixture stirred at room temperature overnight. The mixture was evaporated to dryness and the residue taken up into a mixture of TFA/Dichloromethane (1:1) (10 ml). Allowed to stand at room temperature for 2 h. before being evaporated to dryness. The residue was dissolved in water (20 ml) and the solution was made basic by the addition of 0.88 aqueous ammonia solution. The solid that precipitated was collected by filtration then purified by reverse phase HPLC (25-95% acetonitrile in aqueous ammonia) to afford the title compound (81 mg, 45%).

¹H NMR (300 MHz, DMSO-d₆, 120° C.) δ 8.71 (d, 1H), 8.55 (d, 1H), 8.31 (s, 1H), 8.15 (m, 1H), 7.93 (s, 1H), 7.58 (d, 1H), 7.33 (m, 1H), 6.96 (t, 1H), 5.25 (m, 1H), 4.82 (m, 1H), 3.51 (q, 2H), 2.84 (m, 6H), 2.06 (m, 4H), 1.93 (m, 3H), 1.60 (m, 3H), 1.46 (m, 4H). APCI (Multimode) m/z: 566 [M+H]

### EXAMPLE 2

### 6-(aminomethyl)-N-{cis-4-[6-fluoro-2,4-dioxo-1-(tetrahydro-2H-thiopyran-4-yl)-1,4-dihydropyrido[2,3-d]pyrimidin-3(2H)-yl]cyclohexyl}imidazo[1,2-a]pyridine-2-carboxamide

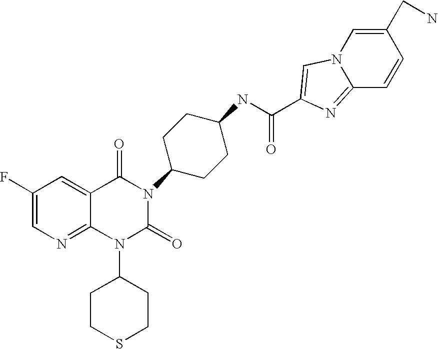

### Step (a) ethyl 6-{[(tert-butoxycarbonyl)amino]methyl}imidazo[1,2-a]pyridine-2-carboxylate

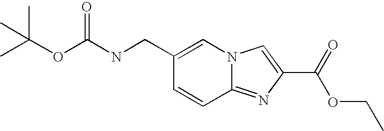

6-Cyano-imidazo[1,2-a]pyridine-2-carboxylic acid ethyl ester (5 g, 23 mmol) was dissolved in ethanol (50 ml) and diterbutyl-dicarbonate (5.08 g, 23 mmol) was added, followed by 10% palladium on carbon (400 mg). The mixture was hydrogenated at 1 bar for 3 hours. The mixture was then filtered and evaporated. The residue was purified by flash chromatography on silica using 5% methanol in DCM as eluent to afford the sub-title compound (340 mg, 4.6%).

¹H NMR (300 MHz, CDCl₃) δ 8.13 (s, 1H), 8.06 (s, 1H), 7.64 (d, 1H), 7.19 (d, 1H), 5.01 (s, 1H), 4.44 (m, 2H), 4.30 (d, 2H), 1.41 (m, 12H)

### Step (b) 6-{[(tert-butoxycarbonyl)amino]methyl}imidazo[1,2-a]pyridine-2-carboxylic acid

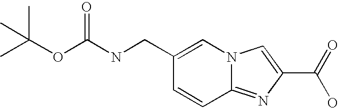

6-(tert-Butoxycarbonylamino-methyl)-imidazo[1,2-a]pyridine-2-carboxylic acid ethyl ester (310 mg, 0.97 mmol) was dissolved in dioxane (10 ml) and lithium hydroxide monohydrate (50 mg, 1.19 mmol) in water (1 ml) was added, and the mixture stirred at room temperature for 4 h. The reaction mixture was evaporated to dryness, the residue redissolved in water (30 ml) and the pH adjusted to 4-5 with glacial acetic acid. The solid that precipitated from solution was collected by filtration and dried to afford the title compound (195 mg, 69%).

¹H NMR (300 MHz, DMSO-d₆) δ 8.55 (s, 1H), 8.34 (s, 1H), 7.63 (d, 1H), 7.46 (s, 1H), 7.25 (d, 1H), 7.25 (d, 1H), 4.12 (d, 2H), 1.40 (s, 9H)

APCI (Multimode) m/z: 292 [M+H]

### Step (c) 6-(aminomethyl)-N-{cis-4-[6-fluoro-2,4-dioxo-1-(tetrahydro-2H-thiopyran-4-yl)-1,4-dihydropyrido[2,3-d]pyrimidin-3(2H)-yl]cyclohexyl}imidazo[1,2-a]pyridine-2-carboxamide

6-(tert-Butoxycarbonylamino-methyl)-imidazo[1,2-a]pyridine-2-carboxylic acid (150 mg, 0.515 mmol) was dissolved in dry DMF (10 ml) and DIEA (0.4 ml, 2.3 mmol) was added, followed by HATU (195 mg, 0.515 mmol). The mixture was stirred at room temperature for 10 min. 3-(4-Amino-cyclohexyl)-6-fluoro-1-(tetrahydro-thiopyran-4-yl)-1H-pyrido[2,3-d]pyrimidine-2,4-dione (195 mg, 0.515 mmol) was added and the mixture stirred at room temperature overnight. The mixture was poured into water (100 ml) and the solid that precipitated was collected by filtration and dried. This solid was then suspended in dioxane (10 ml) and 4.0M hydrogen chloride in dioxane (10 ml) added, and the mixture stirred for 2 h. The reaction was complete so evaporated to dryness and the residue purified by reverse phase HPLC (30-60% acetonitrile in aqeuous ammonia) to afford the title compound (107 mg, 38%).

¹H NMR (300 MHz, DMSO-d₆) δ 8.79 (d, 1H), 8.51 (s, 1H), 8.39 (s, 1H), 8.26 (dd, 1H), 7.74 (d, 1H), 7.65 (d, 1H), 7.38 (dd, 1H), 5.29 (s, 1H), 4.84 (t, 1H), 4.17 (s, 1H), 3.77 (s, 2H), 3.52-3.10 (m, 3H), 2.94-2.65 (m, 7H), 2.10-1.86 (m, 4H), 1.71 (t, 2H), 1.58 (d, 2H)

APCI (Multimode) m/z: 552 [M+H]

### EXAMPLE 3

### 6-(aminomethyl)-N-{cis-4-[6-fluoro-2,4-dioxo-1-(tetrahydro-2H-thiopyran-4-yl)-1,4-dihydropyrido[2,3-d]pyrimidin-3(2H)-yl]cyclohexyl}-5,6,7,8-tetrahydroimidazo[1,2-a]pyridine-2-carboxamide

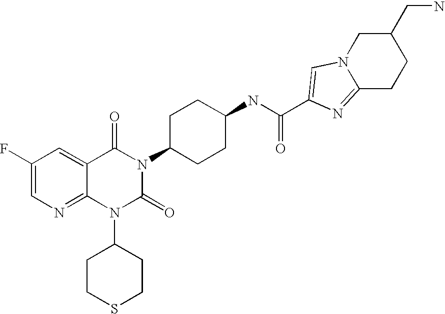

### Step (a) ethyl 6-{[(tert-butoxycarbonyl)amino]methyl}-5,6,7,8-tetrahydroimidazo[1,2-a]pyridine-2-carboxylate

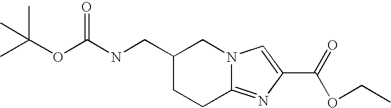

6-Cyano-imidazo[1,2-a]pyridine-2-carboxylic acid ethyl ester (5 g, 23 mmol) was dissolved in ethanol (50 ml), and concentrated hydrochloric acid (1 ml) added, followed by 10% palladium on carbon (500 mg) and the mixture hydrogenated at 1 atmosphere for 2 h. The catalyst was removed by filtration and the pH of the filtrate adjusted to 8-9 by the addition of triethylamine. Diterbutyl-dicarbonate (5.0 g, 23 mmol) was added to the filtrate and the mixture stirred for 8 h. The mixture was then partioned between ethyl acetate and water, and the combined organic extracts were dried (magnesium sulphate), filtered and evaporated. The residue was then purified by flash chromatography on silica using ethyl acetate:dichloromethane (1:1) to afford the sub-title compound (0.45 g, 6%).

### Step (b) 6-{[(tert-butoxycarbonyl)amino]methyl}-5,6,7,8-tetrahydroimidazo[1,2-a]pyridine-2-carboxylic acid

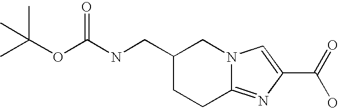

To a solution of 6-(tert-Butoxycarbonylamino-methyl)-5,6,7,8-tetrahydro-imidazo[1,2-a]pyridine-2-carboxylic acid ethyl ester (700 mg, 2.16 mmol) in a mixture of dioxane (20 ml) water (10 ml) was added lithium hydroxide monohydrate (90 mg) and the mixture stirred over night. The mixtures ph was adjusted to 5-6 with glacial acetic acid. The mixture was then evaporated to dryness and the residue taken up into water (no precipitate observed) this solution was then eluted through a C18 silica coated cartridge (10G), washed well with water to remove inorganics, followed by elution with methanol to give the sub-title compound (480 mg).

¹H NMR (400 MHz, DMSO-d₆) δ 7.63 (s, 1H), 7.02 (d, 1H), 4.07 (dd, 1H), 3.57 (m, 2H), 2.79 (m, 2H), 2.00 (m Hz, 2H), 1.36 (m, 9H), 0.88-0.76 (m, 1H), 1.55 (m, 1H)

### Step (c) 6-(aminomethyl)-N-{cis-4-[6-fluoro-2,4-dioxo-1-(tetrahydro-2H-thiopyran-4-yl)-1,4-dihydropyrido[2,3-d]pyrimidin-3(2H)-yl]cyclohexyl}-5,6,7,8-tetrahydroimidazo[1,2-a]pyridine-2-carboxamide

6-{[(tert-butoxycarbonyl)amino]methyl}-5,6,7,8-tetrahydroimidazo[1,2-a]pyridine-2-carboxylic acid (150 mg, 0.515 mmol) was dissolved in dry DMF (10 ml) and DIEA (0.4 ml, 2.3 mmol) was added, followed by HATU (195 mg, 0.515 mmol). The mixture was stirred at room temperature for 10 min. 3-(4-Amino-cyclohexyl)-6-fluoro-1-(tetrahydro-thiopyran-4-yl)-1H-pyrido[2,3-d]pyrimidine-2,4-dione (195 mg, 0.515 mmol) was added and the mixture stirred at room temperature overnight. The mixture was poured into water (100 ml) and the solid that precipitated was collected by filtration and dried. This solid was then suspended in dioxane (10 ml) and 4.0M hydrogen chloride in dioxane (10 ml) added, and the mixture stirred for 2 h. The mixture was evaporated to dryness and the residue purified by reverse phase HPLC (30-60% acetonitrile in aqueous ammonia) to afford the title compound (147 mg, 51%).

¹H NMR (300 MHz, DMSO-d₆) δ 8.78 (d, 1H), 8.24 (dd, 1H), 7.52 (d, 1H), 7.35 (d, 1H), 5.31 (s, 1H), 4.83 (t, 1H), 4.14 (m, 2H), 3.63 (dd, 1H), 2.80 (m, 15H), 2.1-1.88 (m, 6H), 1.70-1.53 (m, 4H)

APCI (Multimode) m/z: 556 [M+H]

The following Examples were prepared in a similar manner to Example 1 step (b) {The compounds were named were named using the index name program from Ogham and stereochemical descriptors added by hand. (See www.eyesopen.com/products/applications/ogham.html)} Where the compounds were isolated as primary or secondary amines the products were prepared by deprotection of a ‘BOC’ group protected using 4M HCl/Dioxane.

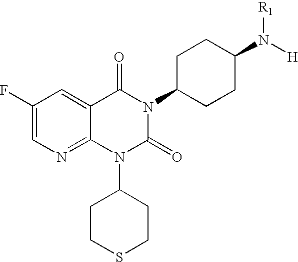

R₁ in the structural fragment presented in the table below shows the point of attachment to the structure directly above.

| Example | | | |
|---|---|---|---|
| No | R₁ | Name | (M + H) |
| 4 | 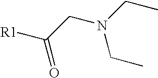 | 2-diethylamino-N-[4-(9-fluoro-2,4-dioxo-5-tetrahydrothiopyran-4-yl-3,5,7-triazabicyclo[4.4.0]deca-6,8,10-trien-3-yl)cyclohexyl]acetamide | 492 |
| 5 | 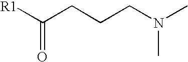 | 4-dimethylamino-N-[4-(9-fluoro-2,4-dioxo-5-tetrahydrothiopyran-4-yl-3,5,7-triazabicyclo[4.4.0]deca-6,8,10-trien-3-yl)cyclohexyl]butanamide | 492 |
| 6 | 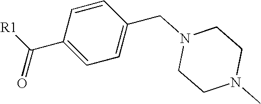 | N-[4-(9-fluoro-2,4-dioxo-5-tetrahydrothiopyran-4-yl-3,5,7-triazabicyclo[4.4.0]deca-6,8,10-trien-3-yl)cyclohexyl]-4-[(4-methylpiperazin-1-yl)methyl]benzamide | 595 |
| 7 | 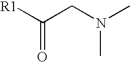 | 2-dimethylamino-N-[4-(9-fluoro-2,4-dioxo-5-tetrahydrothiopyran-4-yl-3,5,7-triazabicyclo[4.4.0]deca-6,8,10-trien-3-yl)cyclohexyl]acetamide | 464 |
| 8 | 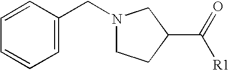 | 1-benzyl-N-[4-(9-fluoro-2,4-dioxo-5-tetrahydrothiopyran-4-yl-3,5,7-triazabicyclo[4.4.0]deca-6,8,10-trien-3-yl)cyclohexyl]pyrrolidine-3-carboxamide | 566 |
| 9 | 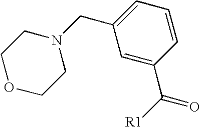 | N-[4-(9-fluoro-2,4-dioxo-5-tetrahydrothiopyran-4-yl-3,5,7-triazabicyclo[4.4.0]deca-6,8,10-trien-3-yl)cyclohexyl]-3-(morpholinomethyl)benzamide | 582 |
| 10 | 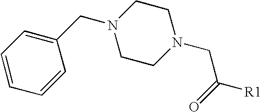 | 2-(4-benzylpiperazin-1-yl)-N-[4-(9-fluoro-2,4-dioxo-5-tetrahydrothiopyran-4-yl-3,5,7-triazabicyclo[4.4.0]deca-6,8,10-trien-3-yl)cyclohexyl]acetamide | 595 |
| 11 | 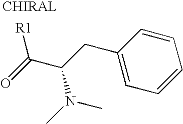 | (2S)-2-dimethylamino-N-[4-(9-fluoro-2,4-dioxo-5-tetrahydrothiopyran-4-yl-3,5,7-triazabicyclo[4.4.0]deca-6,8,10-trien-3-yl)cyclohexyl]-3-phenyl-propanamide | 554 |
| 12 | 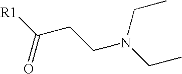 | 3-diethylamino-N-[4-(9-fluoro-2,4-dioxo-5-tetrahydrothiopyran-4-yl-3,5,7-triazabicyclo[4.4.0]deca-6,8,10-trien-3-yl)cyclohexyl]propanamide | 506 |
| 13 | 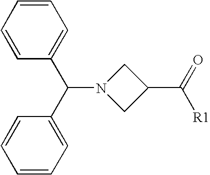 | 1-benzhydryl-N-[4-(9-fluoro-2,4-dioxo-5-tetrahydrothiopyran-4-yl-3,5,7-triazabicyclo[4.4.0]deca-6,8,10-trien-3-yl)cyclohexyl]azetidine-3-carboxamide | 628 |
| 14 | 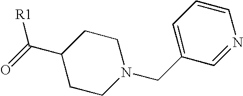 | N-[4-(9-fluoro-2,4-dioxo-5-tetrahydrothiopyran-4-yl-3,5,7-triazabicyclo[4.4.0]deca-6,8,10-trien-3-yl)cyclohexyl]-1-(3-pyridylmethyl)piperidine-4-carboxamide | 581 |
| 15 | 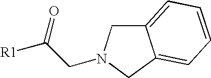 | N-[4-(9-fluoro-2,4-dioxo-5-tetrahydrothiopyran-4-yl-3,5,7-triazabicyclo[4.4.0]deca-6,8,10-trien-3-yl)cyclohexyl]-2-isoindolin-2-yl-acetamide | 538 |
| 16 | 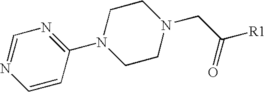 | N-[4-(9-fluoro-2,4-dioxo-5-tetrahydrothiopyran-4-yl-3,5,7-triazabicyclo[4.4.0]deca-6,8,10-trien-3-yl)cyclohexyl]-2-(4-pyrimidin-4-ylpiperazin-1-yl)acetamide | 583 |
| 17 | 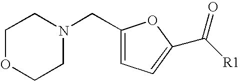 | N-[4-(9-fluoro-2,4-dioxo-5-tetrahydrothiopyran-4-yl-3,5,7-triazabicyclo[4.4.0]deca-6,8,10-trien-3-yl)cyclohexyl]-5-(morpholinomethyl)furan-2-carboxamide | 572 |
| 18 | 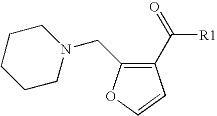 | N-[4-(9-fluoro-2,4-dioxo-5-tetrahydrothiopyran-4-yl-3,5,7-triazabicyclo[4.4.0]deca-6,8,10-trien-3-yl)cyclohexyl]-2-(1-piperidylmethyl)furan-3-carboxamide | 570 |
| 19 | 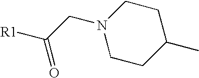 | N-[4-(9-fluoro-2,4-dioxo-5-tetrahydrothiopyran-4-yl-3,5,7-triazabicyclo[4.4.0]deca-6,8,10-trien-3-yl)cyclohexyl]-2-(4-methyl-1-piperidyl)acetamide | 518 |
| 20 | 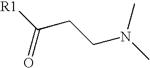 | 3-dimethylamino-N-[4-(9-fluoro-2,4-dioxo-5-tetrahydrothiopyran-4-yl-3,5,7-triazabicyclo[4.4.0]deca-6,8,10-trien-3-yl)cyclohexyl]propanamide | 478 |
| 21 | 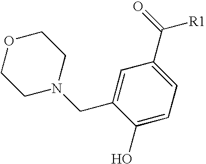 | N-[4-(9-fluoro-2,4-dioxo-5-tetrahydrothiopyran-4-yl-3,5,7-triazabicyclo[4.4.0]deca-6,8,10-trien-3-yl)cyclohexyl]-4-hydroxy-3-(morpholinomethyl)benzamide | 598 |
| 22 | 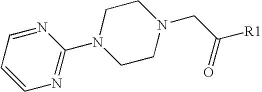 | N-[4-(9-fluoro-2,4-dioxo-5-tetrahydrothiopyran-4-yl-3,5,7-triazabicyclo[4.4.0]deca-6,8,10-trien-3-yl)cyclohexyl]-2-(4-pyrimidin-2-ylpiperazin-1-yl)acetamide | 583 |
| 23 | 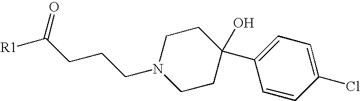 | 4-[4-(4-chlorophenyl)-4-hydroxy-1-piperidyl]-N-[4-(9-fluoro-2,4-dioxo-5-tetrahydrothiopyran-4-yl-3,5,7-triazabicyclo[4.4.0]deca-6,8,10-trien-3-yl)cyclohexyl]butanamide | 658 |
| 24 | 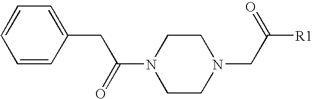 | N-[4-(9-fluoro-2,4-dioxo-5-tetrahydrothiopyran-4-yl-3,5,7-triazabicyclo[4.4.0]deca-6,8,10-trien-3-yl)cyclohexyl]-2-[4-(2-phenylacetyl)piperazin-1-yl]acetamide | 623 |
| 25 | 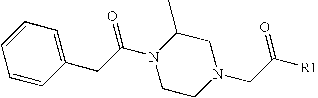 | N-[4-(9-fluoro-2,4-dioxo-5-tetrahydrothiopyran-4-yl-3,5,7-triazabicyclo[4.4.0]deca-6,8,10-trien-3-yl)cyclohexyl]-2-[3-methyl-4-(2-phenylacetyl)piperazin-1-yl]acetamide | 637 |
| 26 | 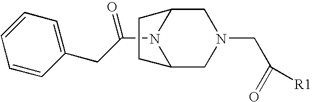 | N-[4-(9-fluoro-2,4-dioxo-5-tetrahydrothiopyran-4-yl-3,5,7-triazabicyclo[4.4.0]deca-6,8,10-trien-3-yl)cyclohexyl]-2-[8-(2-phenylacetyl)-3,8-diazabicyclo[3.2.1]oct-3-yl]acetamide | 649 |
| 27 | 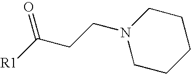 | N-[4-(9-fluoro-2,4-dioxo-5-tetrahydrothiopyran-4-yl-3,5,7-triazabicyclo[4.4.0]deca-6,8,10-trien-3-yl)cyclohexyl]-3-(1-piperidyl)propanamide | 518 |
| 28 | 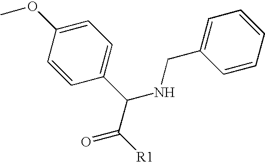 | 2-(benzylamino)-N-[4-(9-fluoro-2,4-dioxo-5-tetrahydrothiopyran-4-yl-3,5,7-triazabicyclo[4.4.0]deca-6,8,10-trien-3-yl)cyclohexyl]-2-(4-methoxyphenyl)acetamide | 632 |
| 29 | 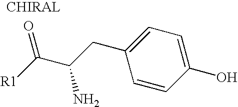 | (2S)-2-amino-N-[4-(9-fluoro-2,4-dioxo-5-tetrahydrothiopyran-4-yl-3,5,7-triazabicyclo[4.4.0]deca-6,8,10-trien-3-yl)cyclohexyl]-3-(4-hydroxyphenyl)propanamide | 542 |
| 30 | 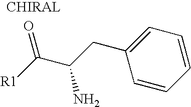 | (2S)-2-amino-N-[4-(9-fluoro-2,4-dioxo-5-tetrahydrothiopyran-4-yl-3,5,7-triazabicyclo[4.4.0]deca-6,8,10-trien-3-yl)cyclohexyl]-3-phenyl-propanamide | 526 |
| 31 | 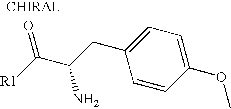 | (2S)-2-amino-N-[4-(9-fluoro-2,4-dioxo-5-tetrahydrothiopyran-4-yl-3,5,7-triazabicyclo[4.4.0]deca-6,8,10-trien-3-yl)cyclohexyl]-3-(4-methoxyphenyl)propanamide | 556 |
| 32 | 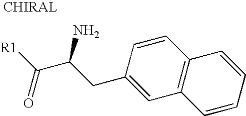 | (2S)-2-amino-N-[4-(9-fluoro-2,4-dioxo-5-tetrahydrothiopyran-4-yl-3,5,7-triazabicyclo[4.4.0]deca-6,8,10-trien-3-yl)cyclohexyl]-3-(2-naphthyl)propanamide | 576 |
| 33 | 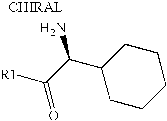 | (2S)-2-amino-2-cyclohexyl-N-[4-(9-fluoro-2,4-dioxo-5-tetrahydrothiopyran-4-yl-3,5,7-triazabicyclo[4.4.0]deca-6,8,10-trien-3-yl)cyclohexyl]acetamide | 518 |
| 34 | 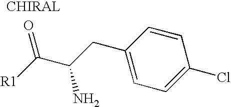 | (2S)-2-amino-3-(4-chlorophenyl)-N-[4-(9-fluoro-2,4-dioxo-5-tetrahydrothiopyran-4-yl-3,5,7-triazabicyclo[4.4.0]deca-6,8,10-trien-3-yl)cyclohexyl]propanamide | 560 |
| 35 | 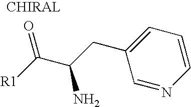 | (2R)-2-amino-N-[4-(9-fluoro-2,4-dioxo-5-tetrahydrothiopyran-4-yl-3,5,7-triazabicyclo[4.4.0]deca-6,8,10-trien-3-yl)cyclohexyl]-3-(3-pyridyl)propanamide | 527 |
| 36 | 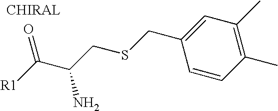 | (2R)-2-amino-3-[(3,4-dimethylphenyl)methylsulfanyl]-N-[4-(9-fluoro-2,4-dioxo-5-tetrahydrothiopyran-4-yl-3,5,7-triazabicyclo[4.4.0]deca-6,8,10-trien-3-yl)cyclohexyl]propanamide | 600 |
| 37 | 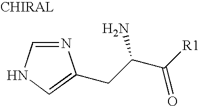 | (2S)-2-amino-N-[4-(9-fluoro-2,4-dioxo-5-tetrahydrothiopyran-4-yl-3,5,7-triazabicyclo[4.4.0]deca-6,8,10-trien-3-yl)cyclohexyl]-3-(1H-imidazol-4-yl)propanamide | 516 |
| 38 | 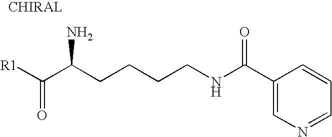 | N-[(5S)-5-amino-5-[[4-(9-fluoro-2,4-dioxo-5-tetrahydrothiopyran-4-yl-3,5,7-triazabicyclo[4.4.0]deca-6,8,10-trien-3-yl)cyclohexyl]carbamoyl]pentyl]pyridine-3-carboxamide | 612 |
| 39 | 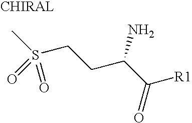 | (2S)-2-amino-N-[4-(9-fluoro-2,4-dioxo-5-tetrahydrothiopyran-4-yl-3,5,7-triazabicyclo[4.4.0]deca-6,8,10-trien-3-yl)cyclohexyl]-4-methylsulfonyl-butanamide | 542 |
| 40 | 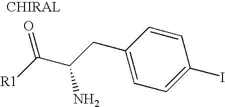 | (2S)-2-amino-N-[4-(9-fluoro-2,4-dioxo-5-tetrahydrothiopyran-4-yl-3,5,7-triazabicyclo[4.4.0]deca-6,8,10-trien-3-yl)cyclohexyl]-3-(4-iodophenyl)propanamide | 652 |
| 41 | 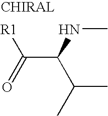 | (2S)-N-[4-(9-fluoro-2,4-dioxo-5-tetrahydrothiopyran-4-yl-3,5,7-triazabicyclo[4.4.0]deca-6,8,10-trien-3-yl)cyclohexyl]-3-methyl-2-methylamino-butanamide | 492 |
| 42 | 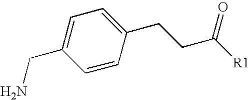 | 3-[4-(aminomethyl)phenyl]-N-[4-(9-fluoro-2,4-dioxo-5-tetrahydrothiopyran-4-yl-3,5,7-triazabicyclo[4.4.0]deca-6,8,10-trien-3-yl)cyclohexyl]propanamide | 540 |
| 43 | 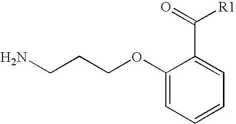 | 2-(3-aminopropoxy)-N-[4-(9-fluoro-2,4-dioxo-5-tetrahydrothiopyran-4-yl-3,5,7-triazabicyclo[4.4.0]deca-6,8,10-trien-3-yl)cyclohexyl]benzamide | 556 |
| 44 | 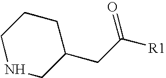 | N-[4-(9-fluoro-2,4-dioxo-5-tetrahydrothiopyran-4-yl-3,5,7-triazabicyclo[4.4.0]deca-6,8,10-trien-3-yl)cyclohexyl]-2-(3-piperidyl)acetamide | 504 |
| 45 | 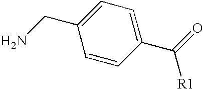 | 4-(aminomethyl)-N-[4-(9-fluoro-2,4-dioxo-5-tetrahydrothiopyran-4-yl-3,5,7-triazabicyclo[4.4.0]deca-6,8,10-trien-3-yl)cyclohexyl]benzamide | 512 |
| 46 | 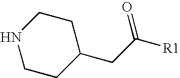 | N-[4-(9-fluoro-2,4-dioxo-5-tetrahydrothiopyran-4-yl-3,5,7-triazabicyclo[4.4.0]deca-6,8,10-trien-3-yl)cyclohexyl]-2-(4-piperidyl)acetamide | 504 |
| 47 | 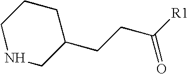 | N-[4-(9-fluoro-2,4-dioxo-5-tetrahydrothiopyran-4-yl-3,5,7-triazabicyclo[4.4.0]deca-6,8,10-trien-3-yl)cyclohexyl]-3-(3-piperidyl)propanamide | 518 |
| 48 | 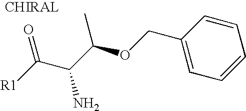 | (2S,3R)-2-amino-3-benzyloxy-N-[4-(9-fluoro-2,4-dioxo-5-tetrahydrothiopyran-4-yl-3,5,7-triazabicyclo[4.4.0]deca-6,8,10-trien-3-yl)cyclohexyl]butanamide | 570 |
| 49 | 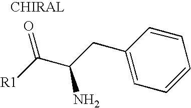 | (2R)-2-amino-N-[4-(9-fluoro-2,4-dioxo-5-tetrahydrothiopyran-4-yl-3,5,7-triazabicyclo[4.4.0]deca-6,8,10-trien-3-yl)cyclohexyl]-3-phenyl-propanamide | 526 |
| 50 | 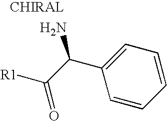 | (2S)-2-amino-N-[4-(9-fluoro-2,4-dioxo-5-tetrahydrothiopyran-4-yl-3,5,7-triazabicyclo[4.4.0]deca-6,8,10-trien-3-yl)cyclohexyl]-2-phenyl-acetamide | 512 |
| 51 | 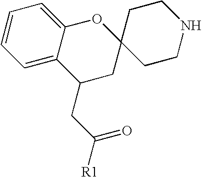 | N-[4-(9-fluoro-2,4-dioxo-5-tetrahydrothiopyran-4-yl-3,5,7-triazabicyclo[4.4.0]deca-6,8,10-trien-3-yl)cyclohexyl]-2-BLAHyl-acetamide | 622 |
| 52 | 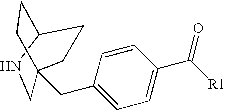 | 4-(8-azabicyclo[2.2.2]oct-1-ylmethyl)-N-[4-(9-fluoro-2,4-dioxo-5-tetrahydrothiopyran-4-yl-3,5,7-triazabicyclo[4.4.0]deca-6,8,10-trien-3-yl)cyclohexyl]benzamide | 606 |
| 53 | 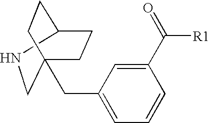 | 3-(8-azabicyclo[2.2.2]oct-1-ylmethyl)-N-[4-(9-fluoro-2,4-dioxo-5-tetrahydrothiopyran-4-yl-3,5,7-triazabicyclo[4.4.0]deca-6,8,10-trien-3-yl)cyclohexyl]benzamide | 606 |
| 54 | 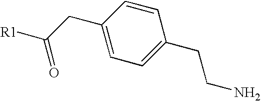 | 2-[4-(2-aminoethyl)phenyl]-N-[4-(9-fluoro-2,4-dioxo-5-tetrahydrothiopyran-4-yl-3,5,7-triazabicyclo[4.4.0]deca-6,8,10-trien-3-yl)cyclohexyl]acetamide | 540 |
| 55 | 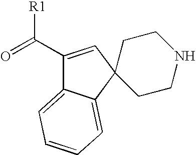 | N-[4-(9-fluoro-2,4-dioxo-5-tetrahydrothiopyran-4-yl-3,5,7-triazabicyclo[4.4.0]deca-6,8,10-trien-3-yl)cyclohexyl]BLAHcarboxamide | 590 |
| 56 | 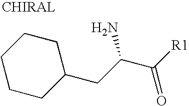 | (2S)-2-amino-3-cyclohexyl-N-[4-(9-fluoro-2,4-dioxo-5-tetrahydrothiopyran-4-yl-3,5,7-triazabicyclo[4.4.0]deca-6,8,10-trien-3-yl)cyclohexyl]propanamide | 532 |
| 57 | 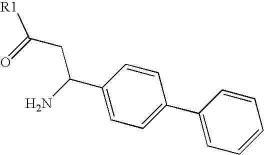 | 3-amino-N-[4-(9-fluoro-2,4-dioxo-5-tetrahydrothiopyran-4-yl-3,5,7-triazabicyclo[4.4.0]deca-6,8,10-trien-3-yl)cyclohexyl]-3-(4-phenylphenyl)propanamide | 602 |
| 58 | 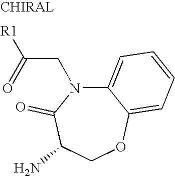 | 2-[(4S)-4-amino-5-oxo-2-oxa-6-azabicyclo[5.4.0]undeca-7,9,11-trien-6-yl]-N-[4-(9-fluoro-2,4-dioxo-5-tetrahydrothiopyran-4-yl-3,5,7-triazabicyclo[4.4.0]deca-6,8,10-trien-3-yl)cyclohexyl]acetamide | 597 |
| 59 | 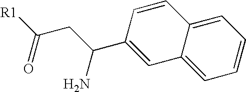 | 3-amino-N-[4-(9-fluoro-2,4-dioxo-5-tetrahydrothiopyran-4-yl-3,5,7-triazabicyclo[4.4.0]deca-6,8,10-trien-3-yl)cyclohexyl]-3-(2-naphthyl)propanamide | 576 |
| 60 | 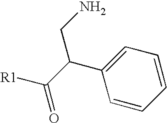 | 3-amino-N-[4-(9-fluoro-2,4-dioxo-5-tetrahydrothiopyran-4-yl-3,5,7-triazabicyclo[4.4.0]deca-6,8,10-trien-3-yl)cyclohexyl]-2-phenyl-propanamide | 526 |
| 61 | 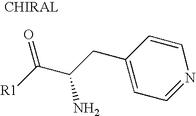 | (2S)-2-amino-N-[4-(9-fluoro-2,4-dioxo-5-tetrahydrothiopyran-4-yl-3,5,7-triazabicyclo[4.4.0]deca-6,8,10-trien-3-yl)cyclohexyl]-3-(4-pyridyl)propanamide | 527 |
| 62 | 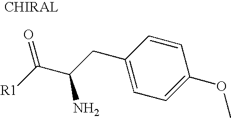 | (2R)-2-amino-N-[4-(9-fluoro-2,4-dioxo-5-tetrahydrothiopyran-4-yl-3,5,7-triazabicyclo[4.4.0]deca-6,8,10-trien-3-yl)cyclohexyl]-3-(4-methoxyphenyl)propanamide | 556 |
| 63 | 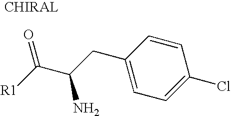 | (2R)-2-amino-3-(4-chlorophenyl)-N-[4-(9-fluoro-2,4-dioxo-5-tetrahydrothiopyran-4-yl-3,5,7-triazabicyclo[4.4.0]deca-6,8,10-trien-3-yl)cyclohexyl]propanamide | 560 |
| 64 | 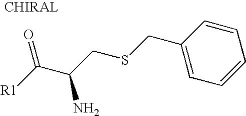 | (2S)-2-amino-3-benzylsulfanyl-N-[4-(9-fluoro-2,4-dioxo-5-tetrahydrothiopyran-4-yl-3,5,7-triazabicyclo[4.4.0]deca-6,8,10-trien-3-yl)cyclohexyl]propanamide | 572 |
| 65 | 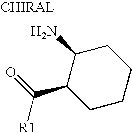 | (1R,2S)-2-amino-N-[4-(9-fluoro-2,4-dioxo-5-tetrahydrothiopyran-4-yl-3,5,7-triazabicyclo[4.4.0]deca-6,8,10-trien-3-yl)cyclohexyl]cyclohexane-1-carboxamide | 504 |
| 66 | 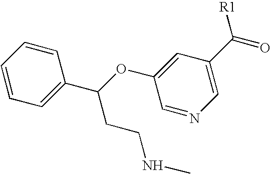 | N-[4-(9-fluoro-2,4-dioxo-5-tetrahydrothiopyran-4-yl-3,5,7-triazabicyclo[4.4.0]deca-6,8,10-trien-3-yl)cyclohexyl]-5-(3-methylamino-1-phenyl-propoxy)pyridine-3-carboxamide | 647 |
| 67 | 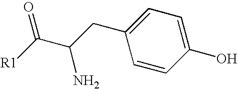 | 2-amino-N-[4-(9-fluoro-2,4-dioxo-5-tetrahydrothiopyran-4-yl-3,5,7-triazabicyclo[4.4.0]deca-6,8,10-trien-3-yl)cyclohexyl]-3-(4-hydroxyphenyl)propanamide | 542 |
| 68 | 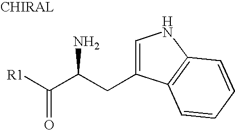 | (2S)-2-amino-N-[4-(9-fluoro-2,4-dioxo-5-tetrahydrothiopyran-4-yl-3,5,7-triazabicyclo[4.4.0]deca-6,8,10-trien-3-yl)cyclohexyl]-3-(1H-indol-3-yl)propanamide | 565 |
| 69 | 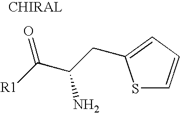 | (2S)-2-amino-N-[4-(9-fluoro-2,4-dioxo-5-tetrahydrothiopyran-4-yl-3,5,7-triazabicyclo[4.4.0]deca-6,8,10-trien-3-yl)cyclohexyl]-3-(2-thienyl)propanamide | 532 |
| 70 | 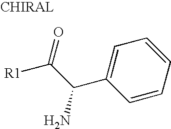 | (2S)-2-amino-N-[4-(9-fluoro-2,4-dioxo-5-tetrahydrothiopyran-4-yl-3,5,7-triazabicyclo[4.4.0]deca-6,8,10-trien-3-yl)cyclohexyl]-2-phenyl-acetamide | 512 |
| 71 | 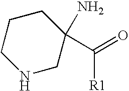 | 3-amino-N-[4-(9-fluoro-2,4-dioxo-5-tetrahydrothiopyran-4-yl-3,5,7-triazabicyclo[4.4.0]deca-6,8,10-trien-3-yl)cyclohexyl]piperidine-3-carboxamide | 505 |
| 72 | 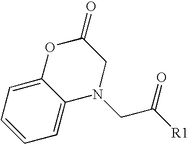 | N-[4-(9-fluoro-2,4-dioxo-5-tetrahydrothiopyran-4-yl-3,5,7-triazabicyclo[4.4.0]deca-6,8,10-trien-3-yl)cyclohexyl]-2-(8-oxo-7-oxa-10-azabicyclo[4.4.0]deca-1,3,5-trien-10-yl)acetamide | 568 |
| 73 | 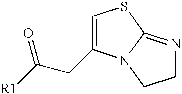 | N-[4-(9-fluoro-2,4-dioxo-5-tetrahydrothiopyran-4-yl-3,5,7-triazabicyclo[4.4.0]deca-6,8,10-trien-3-yl)cyclohexyl]-2-(6-thia-1,4-diazabicyclo[3.3.0]octa-4,7-dien-8-yl)acetamide | 545 |

The following Examples are all Examples of compounds of the invention

| | | | |
|---|---|---|---|
| 74 | 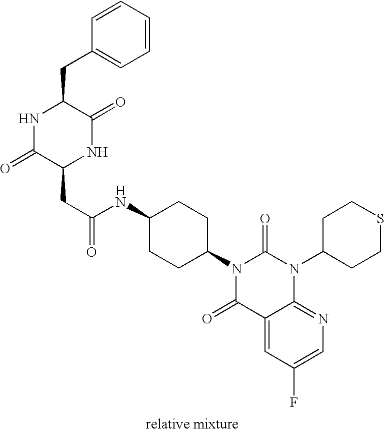 | 2-[(2S,5S)-5-benzyl-3,6-dioxo-piperazin-2-yl]-N-[4-(9-fluoro-2,4-dioxo-5-tetrahydrothiopyran-4-yl-3,5,7-triazabicyclo[4.4.0]deca-6,8,10-trien-3-yl)cyclohexyl]acetamide | 623 |
| 75 | 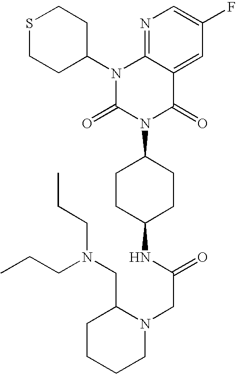 | 2-[2-[(dipropylamino)methyl]-1-piperidyl]-N-[4-(9-fluoro-2,4-dioxo-5-tetrahydrothiopyran-4-yl-3,5,7-triazabicyclo[4.4.0]deca-6,8,10-trien-3-yl)cyclohexyl]acetamide | 617 |
| 76 | 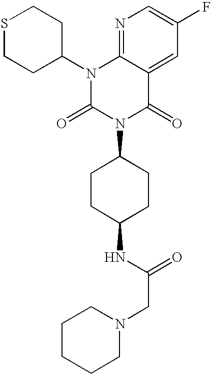 | N-[4-(9-fluoro-2,4-dioxo-5-tetrahydrothiopyran-4-yl-3,5,7-triazabicyclo[4.4.0]deca-6,8,10-trien-3-yl)cyclohexyl]-2-(1-piperidyl)acetamide | 504 |
| 77 | 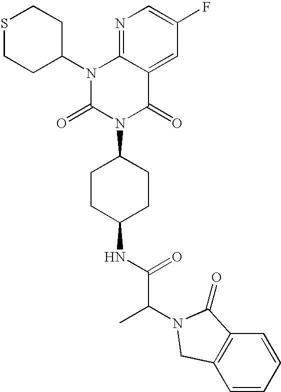 | N-[4-(9-fluoro-2,4-dioxo-5-tetrahydrothiopyran-4-yl-3,5,7-triazabicyclo[4.4.0]deca-6,8,10-trien-3-yl)cyclohexyl]-2-(1-oxoisoindolin-2-yl)propanamide | 566 |
| 78 | 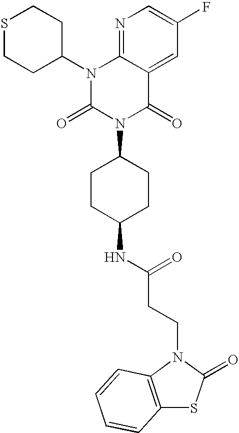 | N-[4-(9-fluoro-2,4-dioxo-5-tetrahydrothiopyran-4-yl-3,5,7-triazabicyclo[4.4.0]deca-6,8,10-trien-3-yl)cyclohexyl]-3-(2-oxobenzothiazol-3-yl)propanamide | 584 |
| 79 | 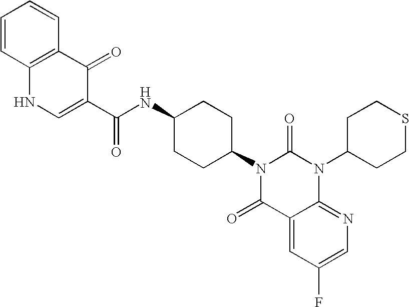 | N-[4-(9-fluoro-2,4-dioxo-5-tetrahydrothiopyran-4-yl-3,5,7-triazabicyclo[4.4.0]deca-6,8,10-trien-3-yl)cyclohexyl]-4-oxo-1H-quinoline-3-carboxamide | 550 |
| 80 | 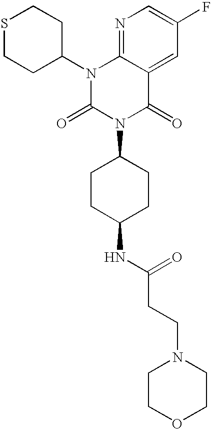 | N-[4-(9-fluoro-2,4-dioxo-5-tetrahydrothiopyran-4-yl-3,5,7-triazabicyclo[4.4.0]deca-6,8,10-trien-3-yl)cyclohexyl]-3-morpholino-propanamide | 520 |
| 81 | 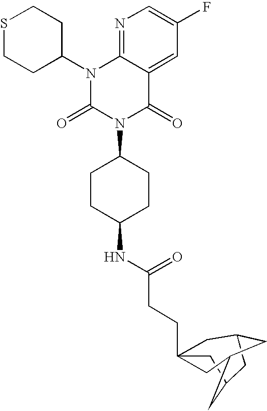 | 3-(1-adamantyl)-N-[4-(9-fluoro-2,4-dioxo-5-tetrahydrothiopyran-4-yl-3,5,7-triazabicyclo[4.4.0]deca-6,8,10-trien-3-yl)cyclohexyl]propanamide | 569 |
| 82 | 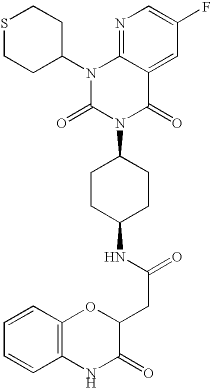 | N-[4-(9-fluoro-2,4-dioxo-5-tetrahydrothiopyran-4-yl-3,5,7-triazabicyclo[4.4.0]deca-6,8,10-trien-3-yl)cyclohexyl]-2-(8-oxo-10-oxa-7-azabicyclo[4.4.0]deca-2,4,11-trien-9-yl)acetamide | 568 |
| 83 | 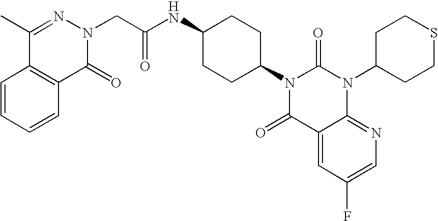 | N-[4-(9-fluoro-2,4-dioxo-5-tetrahydrothiopyran-4-yl-3,5,7-triazabicyclo[4.4.0]deca-6,8,10-trien-3-yl)cyclohexyl]-2-(4-methyl-1-oxo-phthalazin-2-yl)acetamide | 579 |
| 84 | 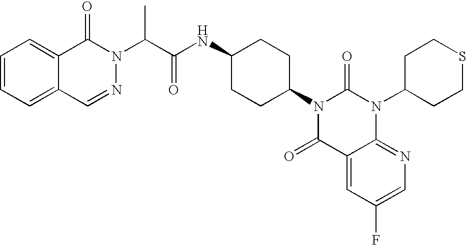 | N-[4-(9-fluoro-2,4-dioxo-5-tetrahydrothiopyran-4-yl-3,5,7-triazabicyclo[4.4.0]deca-6,8,10-trien-3-yl)cyclohexyl]-2-(1-oxophthalazin-2-yl)propanamide | 579 |
| 85 | 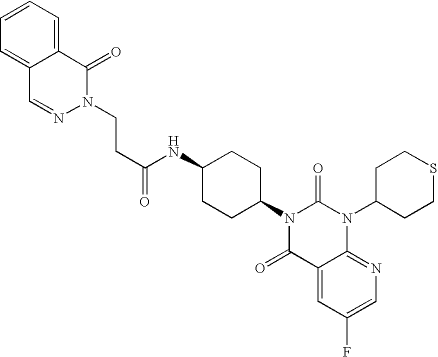 | N-[4-(9-fluoro-2,4-dioxo-5-tetrahydrothiopyran-4-yl-3,5,7-triazabicyclo[4.4.0]deca-6,8,10-trien-3-yl)cyclohexyl]-3-(1-oxophthalazin-2-yl)propanamide | 579 |
| 86 | 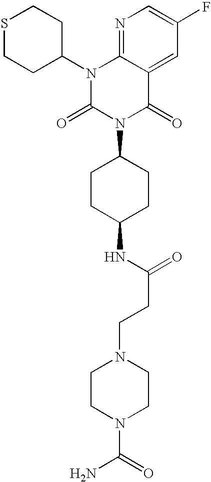 | 4-[2-[[4-(9-fluoro-2,4-dioxo-5-tetrahydrothiopyran-4-yl-3,5,7-triazabicyclo[4.4.0]deca-6,8,10-trien-3-yl)cyclohexyl]carbamoyl]ethyl]piperazine-1-carboxamide | 562 |
| 87 | 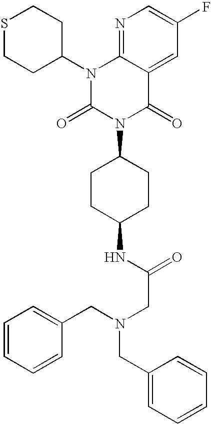 | 2-(dibenzylamino)-N-[4-(9-fluoro-2,4-dioxo-5-tetrahydrothiopyran-4-yl-3,5,7-triazabicyclo[4.4.0]deca-6,8,10-trien-3-yl)cyclohexyl]acetamide | 616 |
| 88 | 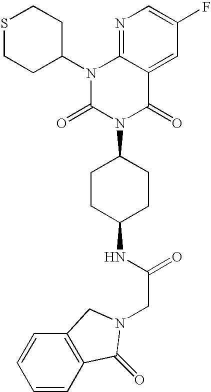 | N-[4-(9-fluoro-2,4-dioxo-5-tetrahydrothiopyran-4-yl-3,5,7-triazabicyclo[4.4.0]deca-6,8,10-trien-3-yl)cyclohexyl]-2-(1-oxoisoindolin-2-yl)acetamide | 552 |
| 89 | 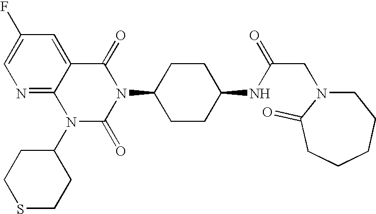 | N-[4-(9-fluoro-2,4-dioxo-5-tetrahydrothiopyran-4-yl-3,5,7-triazabicyclo[4.4.0]deca-6,8,10-trien-3-yl)cyclohexyl]-2-(2-oxoazepan-1-yl)acetamide | 532 |

### EXAMPLE 90

### Human Phosphodiesterase B2 Radiometric Assay

The assay uses recombinant Human Phosphodiesterase B2 (PDE4B2) produced in house (PrAZL0163), stored at −20° C. This assay is based on the observation that 5′AMP, the product of the reaction catalysed by PDE4, binds preferentially to yttrium silicate SPA beads (Amersham Biosciences, UK) compared to the substrate, cAMP. Compounds at the appropriate concentration were preincubated at 30 C for 30 min with an assay buffer containing 50 mM HEPES (pH 7.5), 8.3 mM MgCl2, 1.7 mM EGTA, 0.01% (w/v) Brij® 35 and 0.1 μg/mL recombinant PDE4B2. The reaction was started by the addition of [3H]cyclic AMP to give a final concentration of 8 nM, and was stopped 20 minutes after the addition of the substrate by the addition of yttrium silicate SPA beads containing 18 mM Zn SO4. Bound [3H]cyclic AMP was measured using a Topcount NXT (Packard Bioscience, UK). IC₅₀ values (presented in Table I) were determined using Xlfit3 curve fitting, using model 205.

TABLE I

| Ex | PDE4B2 pIC₅₀ |
|---|---|
| 10 | 10.1 |
| 6 | 8.5 |
| 24 | 8.7 |
| 3 | 8.2 |

1. A compound of formula (I): 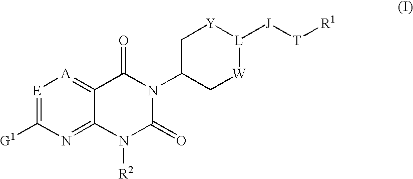
wherein:
E is N or CE¹;
A is N or CA¹;
T is C(O) or S(O)₂;
W is (CH₂)ₙ;
Y is (CH₂)ₚ;
n and p are, independently 0 or 1;
R² is tetrahydrothiopyran-4-yl, tetrahydrothiopyran-4-yl S-oxide, tetrahydrothiopyran-4-yl S-dioxide, tetrahydrothiopyran-4-yl, tetrahydrothiopyran-4-yl S-oxide or tetrahydrothiopyran-4-yl S-dioxide; or aryl or heteroaryl either of which is substituted by one or more of S(O)aryl, S(O)₂aryl, NR²⁵COR²⁶, CONR²⁷R²⁸ (but not C(O)NHaryl), S(O)₂NR²⁹R³⁰ or NRS(O)₂R³¹, and either of which may be additionally optionally substituted by halogen, cyano, hydroxy, C₁₋₄ alkyl, C₁₋₄ alkoxy, CF₃, OCF₃, C₁₋₄ alkylthio, S(O)(C₁₋₄ alkyl), S(O)₂(C₁₋₄ alkyl) or CO₂(C₁₋₄ alkyl);
L is CH or N; when L is CH then J is NH; and when L is N then J is absent and T is bonded directly to L; AND
R¹ is C₁₋₆ alkyl {substituted by either NR³R⁴ or nitrogen-containing heterocyclyl;
and optionally additionally substituted by aryl, heteroaryl, C₃₋₇ cycloalkyl, C₃₋₇ cycloalkyl(C₁₋₄ alkyl), aryl(C₁₋₄ alkoxy), aryl(C₁₋₄ alkylthio), S(O)₂(C₁₋₁₆ alkyl) or NHC(O)heteroaryl}, aryl {substituted by nitrogen-containing heterocyclyl(C₁₋₄ alkyl), amino(C₁₋₄ alkyl), amino(C₁₋₄ alkoxy) or C₁₋₄ alkylamino(C₁₋₄ alkoxy) (itself optionally substituted by phenyl)}, heteroaryl {substituted by nitrogen-containing heterocyclyl(C₁₋₄ alkyl), amino(C₁₋₄ alkyl), amino(C₁₋₄ alkoxy) or C₁₋₄ alkylamino(C₁₋₄ alkoxy) (itself optionally substituted by phenyl)}, nitrogen-containing heterocyclyl {substituted by amino, aryl(C₁₋₄ alkyl) or heteroaryl(C₁₋₄ alkyl)}, aryl(C₁₋₄ alkyl) {substituted by amino(C₁₋₄ alkyl)} or C₃₋₇ cycloalkyl {substituted by nitrogen-containing heterocyclyl or amino};
provided that when R² is aryl or heteroaryl, each of which is optionally substituted by halogen, cyano, hydroxy, C₁₋₄ alkyl, C₁₋₄ alkoxy, CF₃, OCF₃, C₁₋₄ alkylthio, S(O)(C₁₋₄ alkyl), S(O)₂(C₁₋₄ alkyl) or C(O)₂(C₁₋₄ alkyl); then R¹ is not C₁₋₆ alkyl substituted by nitrogen-containing heterocyclyl;
OR
L is CH and J is N(CH₂)ₘR⁹⁹; m is 1, 2, 3 or 4; R⁹⁹ is NH₂, phenyl or heteroaryl;
AND
R¹ is C₁₋₆ alkyl {optionally substituted by hydroxyl, C₁₋₆ alkoxy, NR⁷⁷R⁸⁸, heterocyclyl (optionally substituted by oxo, hydroxy, C₁₋₆ alkyl, aryl, heteroaryl, aryl(C₁₋₄ alkyl), heterocyclyl or C(O)(C₁₋₄ alkyl)phenyl), aryl, heteroaryl, C₃₋₇ cycloalkyl, C₃₋₇ cycloalkyl(C₁₋₄ alkyl), CO₂H, CO₂(C₁₋₆ alkyl), aryl(C₁₋₄ alkoxy), aryl(C₁₋₄ alkylthio), S(O)₂(C₁₋₆ alkyl), NHC(O)heteroaryl or NHC(O)R⁶⁶}, C₁₋₆ alkoxy, C₃₋₆ cycloalkyl (optionally substituted by hydroxyl or C₁₋₆ alkyl), heterocyclyl {optionally substituted by oxo, hydroxy, C₁₋₆ alkyl, amino, aryl, heteroaryl, aryl(C₁₋₄ alkyl), heteroaryl(C₁₋₄ alkyl), heterocyclyl or C(O)(C₁₋₄ alkyl)phenyl}, aryl(C₁₋₄ alkyl) {substituted by amino(C₁₋₄ alkyl)}, aryl or heteroaryl;
R⁶⁶ is C₁₋₆ alkyl or phenyl;
R³, R⁴, R⁷⁷ and R⁸⁸ are, independently, hydrogen, C₁₋₆ alkyl or phenyl(C₁₋₄ alkyl);
in addition to any substituents that might be specified above the foregoing nitrogen-containing heterocyclyl rings are optionally substituted by oxo, hydroxy, C₁₋₆ alkyl (itself optionally substituted by NH₂, NH(C₁₋₄ alkyl) or N(C₁₋₄ alkyl)₂), NH₂, aryl, heteroaryl, aryl(C₁₋₄ alkyl), heteroaryl(C₁₋₄ alkyl), heterocyclyl or C(O)(C₁₋₄ alkyl)phenyl;
in addition to any required substituents that might be specified above the foregoing phenyl, aryl and heteroaryl moieties are, independently, optionally substituted by:
halogen, cyano, nitro, hydroxy, S(O)_{q}R²⁴, OC(O)NR⁵R⁶, NR⁷R⁸, NR⁹C(O)R¹⁰, NR¹¹C(O)NR¹²R¹³, S(O)₂NR¹⁴R¹⁵, NR¹⁶S(O)₂R¹⁷, C(O)NR¹⁸R¹⁹, C(O)R²⁰, CO₂R²¹, NR²²CO₂R²³, C₁₋₆ alkyl, C₁₋₆ hydroxyalkyl, C₁₋₆ haloalkyl, C₁₋₆ alkoxy(C₁₋₆)alkyl, di(C₁₋₆)alkylamino(C₁₋₆)alkyl, C₁₋₆ alkoxy, C₁₋₆ haloalkoxy, C₁₋₆ alkoxy(C₁₋₆)alkoxy, C₁₋₆ alkylthio, C₂₋₆ alkenyl, C₂₋₆ alkynyl, C₃₋₁₀ cycloalkyl (itself optionally substituted by C₁₋₄ alkyl or oxo), methylenedioxy, difluoromethylenedioxy, phenyl, phenyl(C₁₋₄)alkyl, phenoxy, phenylthio, phenyl(C₁₋₄)alkoxy, heteroaryl, heteroaryl(C₁₋₄)alkyl, heteroaryloxy or heteroaryl(C₁₋₄)alkoxy; wherein any of the immediately foregoing phenyl and heteroaryl moieties are optionally substituted with halogen, hydroxy, nitro, S(O)ᵣ(C₁₋₄ alkyl), S(O)₂NH₂, S(O)₂NH(C₁₋₄ alkyl), S(O)₂N(C₁₋₄ alkyl)₂, cyano, C₁₋₄ alkyl, C₁₋₄ alkoxy, C(O)NH₂, C(O)NH(C₁₋₄ alkyl), C(O)N(C₁₋₄ alkyl)₂, CO₂H, CO₂(C₁₋₄ alkyl), NHC(O)(C₁₋₄ alkyl), NHS(O)₂(C₁₋₄ alkyl), C(O)(C₁₋₄ alkyl), CF₃ or OCF₃;
A¹, E¹ and G¹ are, independently, hydrogen, halogen, cyano, hydroxy, C₁₋₄ alkyl, C₁₋₄ alkoxy, CF₃ or OCF₃;
q and r are, independently, 0, 1 or 2;
R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵, R²⁶, R²⁷, R²⁸, R²⁹, R³⁰ and R³¹ are, independently, C₁₋₆ alkyl {optionally substituted by halogen, hydroxy or C₁₋₆ alkoxy}, CH₂(C₂₋₆ alkenyl), phenyl {itself optionally substituted by halogen, hydroxy, nitro, NH₂, NH(C₁₋₄ alkyl), N(C₁₋₄ alkyl)₂, S(O)₂(C₁₋₄ alkyl), S(O)₂NH₂, S(O)₂NH(C₁₋₄ alkyl), S(O)₂N(C₁₋₄ alkyl)₂, cyano, C₁₋₄ alkyl, C₁₋₄ alkoxy, C(O)NH₂, C(O)NH(C₁₋₄ alkyl), C(O)N(C₁₋₄ alkyl)₂, CO₂H, CO₂(C₁₋₄ alkyl), NHC(O)(C₁₋₄ alkyl), NHS(O)₂(C₁₋₄ alkyl), C(O)(C₁₋₄ alkyl), CF₃ or OCF₃} or heteroaryl {itself optionally substituted by halogen, hydroxy, nitro, NH₂, NH(C₁₋₄ alkyl), N(C₁₋₄ alkyl)₂, S(O)₂(C₁₋₄ alkyl), S(O)₂NH₂, S(O)₂NH(C₁₋₄ alkyl), S(O)₂N(C₁₋₄ alkyl)₂, cyano, C₁₋₄ alkyl, C₁₋₄ alkoxy, C(O)NH₂, C(O)NH(C₁₋₄ alkyl), C(O)N(C₁₋₄ alkyl)₂, CO₂H, CO₂(C₁₋₄ alkyl), NHC(O)(C₁₋₄ alkyl), NHS(O)₂(C₁₋₄ alkyl), C(O)(C₁₋₄ alkyl), CF₃ or OCF₃};
R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵, R²⁶, R²⁷, R²⁸, R²⁹ and R³⁰ can also be hydrogen;
or a N-oxide thereof, or a pharmaceutically acceptable salt thereof.

2. A compound of formula (I) as claimed in claim 1 wherein E is CE¹, and E¹ is hydrogen or fluoro.

3. A compound of formula (I) as claimed in claim 1 or 2 wherein A is CA¹, and A¹ is hydrogen.

4. A compound of formula (I) as claimed in claim 1, 2 or 3 wherein G¹ is hydrogen.

5. A compound of formula (I) as claimed in claim 1, 2, 3 or 4 wherein n and p are both 1.

6. A compound of formula (I) as claimed in any preceding claim wherein L is CH.

7. A compound of formula (I) as claimed in any preceding claim wherein T is C(O).

8. A compound of formula (I) as claimed in any preceding claim wherein J is NH.

9. A compound of formula (I) as claimed in claim 1, 2, 3 or 4 wherein Y and W are both CH₂, L is CH, J is NH and T is C(O).

10. A compound of formula (I) as claimed in any preceding claim wherein L is CH, J is NH; and R¹ is C₁₋₆ alkyl {substituted by either NR³R⁴ or nitrogen-containing heterocyclyl; and optionally additionally substituted by aryl, heteroaryl, C₃₋₇ cycloalkyl, C₃₋₇ cycloalkyl(C₁₋₄ alkyl), aryl(C₁₋₄ alkoxy), aryl(C₁₋₄ alkylthio), S(O)₂(C₁₋₆ alkyl) or NHC(O)heteroaryl}, aryl {substituted by nitrogen-containing heterocyclyl(C₁₋₄ alkyl), amino(C₁₋₄ alkyl), amino(C₁₋₄ alkoxy) or C₁₋₄ alkylamino(C₁₋₄ alkoxy) (itself optionally substituted by phenyl)}, heteroaryl {substituted by nitrogen-containing heterocyclyl(C₁₋₄ alkyl), amino(C₁₋₄ alkyl), amino(C₁₋₄ alkoxy) or C₁₋₄ alkylamino(C₁₋₄ alkoxy) (itself optionally substituted by phenyl)}, nitrogen-containing heterocyclyl {substituted by amino, aryl(C₁₋₄ alkyl) or heteroaryl(C₁₋₄ alkyl)}, aryl(C₁₋₄ alkyl) {substituted by amino(C₁₋₄ alkyl)} or C₃₋₇ cycloalkyl {substituted by nitrogen-containing heterocyclyl or amino}; nitrogen-containing heterocyclyl is optionally substituted by C₁₋₄ alkyl, NH₂, oxo, hydroxy, aryl, heteroaryl, aryl(C₁₋₄ alkyl) or C(O)(C₁₋₄ alkyl)phenyl; R³ and R⁴ are as defined above; provided that when R² is aryl or heteroaryl, each of which is optionally substituted by halogen, cyano, hydroxy, C₁₋₄ alkyl, C₁₋₄ alkoxy, CF₃, OCF₃, C₁₋₄ alkylthio, S(O)(C₁₋₄ alkyl), S(O)₂(C₁₋₄ alkyl) or C(O)₂(C₁₋₄ alkyl); then R¹ is not C₁₋₆ alkyl substituted by nitrogen-containing heterocyclyl.

11. A compound of formula (I) as claimed in any preceding claim wherein L is CH, J is NH; and R¹ is C₁₋₆ alkyl {substituted by NR³R⁴; and optionally additionally substituted by aryl, heteroaryl, C₃₋₇ cycloalkyl, C₃₋₇ cycloalkyl(C₁₋₄ alkyl), aryl(C₁₋₄ alkoxy), aryl(C₁₋₄ alkylthio), S(O)₂(C₁₋₆ alkyl) or NHC(O)heteroaryl}; wherein R³ and R⁴ are as defined above.

12. A compound of formula (I) as claimed in any preceding claim wherein R² is tetrahydrothiopyranyl.

13. A process for preparing a compound of formula (I) as claimed in claim 1, the process comprising:
performing the following reaction using literature conditions: 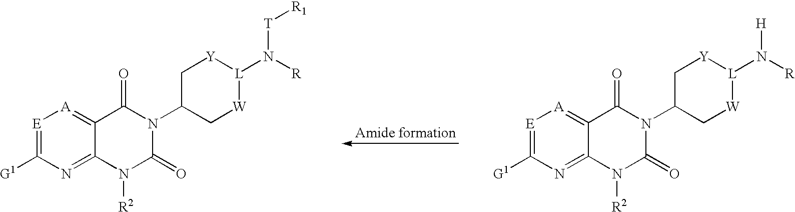
wherein T is C(O), and Rhydrogen or (CH₂)ₘR⁹⁹; or,
removing the Boc protecting group from a compound of formula (II) 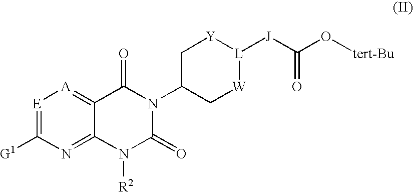
wherein m, A, R², G¹, E, Y, L, W, and J are as defined in claim 1, and reacting the product so formed with a carboxylic acid of formula (III): 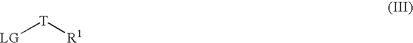
wherein R¹ and T are as defined in claim 1, and LG is a is a leaving group.

14. A pharmaceutical composition which comprises a compound of the formula (I), or a pharmaceutically acceptable salt thereof as claimed in claim 1, and a pharmaceutically acceptable adjuvant, diluent or carrier.

15. A compound of the formula (I), or a pharmaceutically acceptable salt thereof as claimed in claim 1, for use in therapy.

16. A compound of formula (I), or a pharmaceutically acceptable salt thereof as claimed in claim 1, in the manufacture of a medicament for use in therapy.

17. A method of treating a PDE 4 mediated disease state in a mammal suffering from, or at risk of, said disease, which comprises administering to a mammal in need of such treatment a therapeutically effective amount of a compound of formula (I), or a pharmaceutically acceptable salt thereof as claimed in claim 1.

18. A compound of formula (I), or a pharmaceutically acceptable salt thereof as claimed in claim 1, for the treatment of a PDE 4 mediated disease state.

19. A pharmaceutical product comprising, in combination, a first active ingredient which is a compound of formula (I), or a pharmaceutically acceptable salt thereof, as hereinbefore described, and at least one further active ingredient selected from
a β2. adrenoceptor agonist,
a modulator of chemokine receptor function,
an inhibitor of kinase function,
a protease inhibitor,
a steroidal glucocorticoid receptor agonist,
an anticholinergic agent, and a
a non-steroidal glucocorticoid receptor agonist.
